(19) United States
(12) Patent Application Publication
(10) Pub. No.: US 2024/0376172 A1
(43) Pub. Date: Nov. 14, 2024

(54) INTERLEUKIN 2 CHIMERIC CONSTRUCTS WITH TARGETING SPECIFICY TO INFLAMED TISSUES

(71) Applicants: ILTOO PHARMA, Paris (FR); SORBONNE UNIVERSITE, Paris (FR); ASSISTANCE PUBLIQUE - HÔPITAUX DE PARIS, Paris (FR); INSERM (INSTITUT NATIONAL DE LA SANTÉ ET DE LA RECHERCHE MÉDICALE), Paris (FR)

(72) Inventors: David KLATZMANN, Paris (FR); Alain TEDGUI, Paris (FR); Thomas VAZQUEZ, Montreuil (FR); Nicolas BILLIALD, Paris (FR)

(21) Appl. No.: 18/698,994

(22) PCT Filed: Oct. 6, 2022
(86) PCT No.: PCT/EP2022/077847

§ 371 Date: Apr. 5, 2024

(30) Foreign Application Priority Data
Oct. 6, 2021 (EP) 21306399.3

(51) Int. Cl. C07K 14/55; A61K 38/00; A61P 29/00; A61P 37/06; C07K 14/47; C07K 16/44; C12N 15/86

(52) U.S. Cl. CPC C07K 14/55; A61P 29/00; A61P 37/06; C07K 14/472; C07K 16/44; C12N 15/86; A61K 38/00; C07K 2317/56; C07K 2317/622; C07K 2319/01; C12N 2740/15043

(57) ABSTRACT

The present invention relates to a targeted chimeric construct, comprising i) an interleukin 2 (IL2) moiety and ii) a targeting moiety which binds to an oxidized protein or oxidized lipid. The targeting moiety is preferably an antibody or scFv binding specific oxidized proteins or oxidized lipids and targets the fusion protein to inflammatory tissues. The chimeric construct preferably further comprises a beta chain of the C4b-binding protein (C4BP), which is capable of forming a dimeric protein.

The present invention relates to chimeric constructs comprising IL2 and a targeting moiety that provides specificity to inflamed tissues.

### BACKGROUND OF THE INVENTION

Interleukin-2 (IL2 or IL-2) is a cytokine that regulates key aspects of the immune system. IL2 has been used in attempts to boost immune responses in patients with cancer, as well as autoimmune and/or inflammatory diseases. IL2 is a potent T cells growth factor that promotes immune responses, including clonal expansion of antigen-activated T cells, drives development of CD4+ T-helper (Th)1 and Th2 cells, terminally differentiates CD8+ cytotoxic T lymphocytes (CTLs), and opposes development of CD4+ Th17 and T-follicular helper (Tfh) cells. IL2 also shapes T cell memory recall responses.

Low doses of IL2 have been used to selectively boost tolerance to suppress unwanted immune responses associated with autoimmune-like attack of self-tissues. The experience thus far has been that this therapy is safe, with no indication of reactivation of auto-aggressive T cells, while regulatory T cells (Tregs) increase in nearly all patients, which is accompanied by clinical improvement.

Nevertheless, IL2 as a therapeutic can be improved. Notably, it is desirable to improve the efficacy of the treatment by targeting IL2 to the tissue of interest, thereby increasing the concentrations locally, at the tissue of interest. There is also a need to provide IL2 constructs with targeting specify in order to limit the potential “off-target” effects related to the ubiquitous presence of lymphocyte populations capable of being activated in response to IL-2.

### SUMMARY OF THE INVENTION

The invention provides an IL2 chimeric construct with targeting specificity to inflammatory tissues.

The inventors have surprisingly shown that such targeted chimeric constructs significantly improve the efficacy of the IL2 treatment. In particular, the present inventors have shown that the use of the targeted chimeric constructs of the invention leads to an increased number of Treg recruited to the inflammatory site, and to an increased proliferation of Tregs.

Specifically, the chimeric constructs of the invention comprise (i) at least one interleukin 2 moiety; and ii) at least one targeting moiety, which binds to an oxidized protein or oxidized lipid, such as a pro-inflammatory oxidized protein or oxidized lipid.

In a preferred embodiment, the targeting moiety binds to an oxidation-specific epitope (OSE). In a particular embodiment, the targeting moiety binds to (i) a malondialdehyde (MDA) epitope, (ii) to a 2-(ω-carboxyethyl) pyrrole (CEP) epitope, (iii) to a 4-hydroxynonenal (4-HNE) epitope, or to (iv) an oxidized phospholipid (OxPL), such as a phosphocholine-containing oxidized phospholipid (PC-OxPL), an oxidized phosphatidylethanolamine (OxPE), an oxidized phosphatidylserine (OxPS) or an oxidized cardiolipin (OxCL), preferably to a phosphocholine-containing oxidized phospholipid (PC-OxPL).

In a particular embodiment, the targeting moiety is an antibody or an antibody fragment, such as a single chain variable fragment (scFv).

In a particular embodiment, the targeting moiety is selected from the group consisting of: a E06 antibody or a E06 antibody fragment such as a E06 scFv; a LR04 antibody or a LR04 antibody fragment such as a LR04 scFv; a NA17 antibody or a NA17 antibody fragment such as a NA17 scFv; a E014 antibody or a E014 antibody fragment such as a E014 scFv; a MDA2 antibody or a MDA2 antibody fragment such as a MDA2 scFv; a IK17 antibody or a IK17 antibody fragment such as a IK17 scFv; a LR01 antibody or a LR01 antibody fragment such as LR01 scFv; and functional variants thereof.

In a preferred embodiment, the targeting moiety is a E06 antibody or a E06 antibody fragment such as a E06 scFv, or a functional variant thereof. In a particular embodiment, the E06 scFv comprises:
a variable heavy chain (VH) domain comprising an amino acid sequence as shown in SEQ ID NO: 12, or an amino acid sequence having at least 75%, 80%, 85%, 90%, 95%, 96%, 97%, 98% or at least 99% identity with SEQ ID NO:12; and
a variable light chain (VL) domain comprising an amino acid sequence as shown in SEQ ID NO: 11, or an amino acid sequence having at least 75%, 80%, 85%, 90%, 95%, 96%, 97%, 98% or at least 99% identity with SEQ ID NO:11.

In a particular embodiment, the IL-2 moiety is human IL-2 or homologous variant thereof, wherein the variant has at least 85% amino acid identity with human wild-type IL-2, preferably wherein the variant is an active analogue of human IL-2 which has at least 90% amino acid identity with human wild-type IL-2, wherein said IL-2 moiety is preferably an IL2 mutein that comprises a substitution at position N88 of SEQ ID NO: 2, still preferably substitution N88R or N88D.

In a particular embodiment, the IL2 moiety and the targeting moiety are fused in frame or through an amino acid linker, preferably a polyG linker.

In a particular embodiment, said chimeric construct further comprises a beta chain of the C4b-binding protein (C4BPβ) or at least one fragment or functional variant thereof that is capable of forming a dimeric protein.

In a particular embodiment, the fragment of C4BPβ comprises, or consists of, amino acid residues 194 to 252 of C4BPβ or a longer fragment of C4BPβ that extends at the N-term up to at most amino acid 135.

In a particular embodiment, the chimeric construct comprises a functional variant of C4BPβ which comprises:
a) a modified sequence of the fragment of C4BPβ, wherein less than 25 percent of the amino acids of the fragment, preferably less than 10 percent, have been cut out or replaced, in which the cysteines located in positions 202 and 216 as well as at least 3 amino acids upstream and downstream of each cysteine have been conserved; or
b) a modified sequence of the fragment of the C4BPβ, wherein a cysteine responsible for dimerization is substituted with an amino acid, preferably selected from alanine, valine, phenylalanine, proline, methionine, isoleucine, leucine and tryptophan, and another amino acid of the fragment is substituted with a cysteine; or
c) a sequence of the fragment of C4BPβ modified by insertion of a sequence which is heterologous to the beta chain, between the cysteines responsible for dimerization; or
d) a sequence of the fragment of C4BPβ modified by cutting out amino acids between the cysteines responsible for dimerization.

In a particular embodiment, the IL-2 moiety is fused at the N-terminus of C4BPβ or said fragment thereof, wherein the C-terminus of C4BPβ or said fragment thereof is preferably fused to the targeting moiety.

In a particular embodiment, the chimeric construct is in dimer form, wherein the monomers are associated by covalent bonding between two cysteines of C4BPβ. Homodimers and heterodimers are described in greater details below.

Another aspect of the invention relates to a nucleic acid encoding the chimeric construct of the invention. The invention also relates to a vector comprising said nucleic acid, and to a host cell comprising said nucleic acid or said vector.

The invention also relates to the chimeric construct of the invention, for use in treating an auto-immune and/or inflammatory disease.

### LEGENDS TO THE FIGURES

FIG. 1: In vitro evaluation of the functional design of the targeted fusion proteins.

Stable HEK 293T cell lines transduced with lentiviral vector containing transgenes coding for each of the targeted fusion proteins were put in cultured for 48H in a serum-free medium.

A) Design of the targeted fusion proteins

B) Targeted fusion proteins detection in stable HEK 293T cells supernatants by enzyme-linked immunosorbent assay (ELISA).

C) Using concentrated and filtered supernatants, IL2-C4bpß-scFv and IL2N88R-C4bpß-scFv were then characterized by Western blot using either a primary polyclonal anti-human IL-2 antibody or a primary monoclonal anti-histidine antibody.

D) The functional activities of the targeted fusion proteins were assessed by evaluation of the STAT5 phosphorylation (pSTAT5) response in human CD4+ regulatory T cells, CD4+ conventional T cells, CD8+ T cells and NK cells using flow cytometry.

FIG. 2: Therapeutic effects of the targeted fusion proteins in colitis inflammatory experimental model.

A) Six to eight-weeks-old C57BL/6 (Jrj) female mice (n=6) were injected by intraperitoneal route with 5·10¹⁰ to 5·10¹¹ vg (viral genome) of AAV coding for IL2, scFvE06, IL2-C4bpß-scFvE06 or IL2_{N88R}-C4bpß-scFvE06 or left untreated seven days before immunization by oral administration of dextran sulfate sodium (2%) diluted in water during 6 days. Immunomonitoring analysis of CD4+ Treg and CD25 MFI, CD4+ Tconv CD25+ and NK cells in peripheral blood seven days after AAV injections were performed (B).

Clinical disease evaluation is based on the weight loss (C), the stool consistency (D), haemorrhage (E) and the disease activity index (F). Immunomonitoring analysis of Tregs expressing integrin α4β7 (G) and Ki67 (H) in brachial and para-aortic lymph nodes. Statistical significances were evaluated between different groups of treatment after calculation of AUC using GraphPad Prism version 6.00 and calculated using the Mann-Whitney test (comparison of means, unpaired test, nonparametric test, two-tail P value) with P<0.05 (*) taken as statistical significance (** P<0.01, *** P<0.001). For all the graphs, error bars represent Standard Error of the Mean (SEM).

FIG. 3 is a graph that reports pharmacokinetics in mice after a single injection of IL2 or IL2-C4bpß-scFvE06.

FIG. 4 is a group of photos of immunochemistry staining in a DSS-induced colitis mice model. Only mice treated with IL-2-C4bpß-scFvE06 showed detection of both IL2 and 6×-HisTag in colon.

### DETAILED DESCRIPTION OF THE INVENTION

### Definitions

The “subject” or “patient” to be treated may be any mammal, preferably a human being. The human subject may be a child, an adult or an elder.

The term “treating” or “treatment” means any improvement in the disease. It includes alleviating at least one symptom, or reducing the severity or the development of the disease. When the disease is an inflammatory and/or autoimmune disorder, the term more particularly includes reducing the risk, occurrence or severity of acute episodes (flares). The term “treating” or “treatment” encompasses reducing the progression of the disease. In particular the invention encompasses preventing or slowing down the progression of the disease. The term “treating” or “treatment” further encompasses prophylactic treatment, by reducing the risk or delaying the onset of the disease, especially in a subject who is asymptomatic but has been diagnosed as being “at risk”.

“Regulatory T cells” or “Tregs” are T lymphocytes having immunosuppressive activity. Natural Tregs are characterized as CD4+CD25+Foxp3+ cells. Tregs play a major role in the control of inflammatory diseases, although their mode of action in such disease is not well understood. In fact, in most inflammatory diseases, Treg depletion exacerbates disease while Treg addition decreases it. Most Tregs are CD4+ cells, although there also exists a rare population of CD8+Foxp3+ T lymphocytes with a suppressive activity.

Within the context of this application, “effector T cells” (or “Teff”) designates conventional T lymphocytes other than Tregs (sometimes also referred to as Tconv in the literature), which express one or more T cell receptor (TCR) and perform effector functions (e.g., cytotoxic activity, cytokine secretion, etc). Major populations of human Teff according to this invention include CD4+ T helper lymphocytes (e.g., Th0, Th1, Th2, Th9, Th17, Tfh) and CD4+ or CD8+ cytotoxic T lymphocytes, and they can be specific for self or non-self antigens. Teff does not comprise the Foxp3+ regulatory CD8+ T cells.

Within the context of this application, “T follicular helper cells” (or “Tfh”) designates T CD4+ lymphocytes that express BcL6, CXCR5 and PD1, are Foxp3−, and provide B cell help.

Within the context of this application, “T follicular regulatory cells” (or “Tfr”) designates CD4+CXCR5+PD-1+Bcl6+Foxp3+CD25− T lymphocytes.

An antibody “specifically binds” to a target antigen if it binds with greater affinity, avidity, more readily, and/or with greater duration than it binds to other substances. “Specific binding” or “preferential binding” does not necessarily require (although it can include) exclusive binding. Generally, but not necessarily, reference to binding means preferential binding. A variety of methods of measuring binding affinity are known in the art, any of which can be used for purposes of this disclosure.

“Antibody fragments” comprise only a portion of an antibody, wherein the portion typically retains at least one, more commonly most or all, of the functions normally associated with that portion when present in the intact antibody. Examples of antibody fragments include Fab, Fab′, F(ab′)2, and Fv fragments; diabodies; linear antibodies; single-chain antibody molecules; and multispecific antibodies formed from antibody fragments. In one embodiment, an antibody fragment comprises an antigen binding site of the original antibody and thus retains the ability to bind antigen. In another embodiment, an antibody fragment, for example one that comprises the Fc region, retains at least one of the biological functions normally associated with the Fc region when present in the original antibody, such as FcRn binding, antibody half-life modulation, ADCC function and complement binding.

The antigen-binding regions or antigen-binding fragments correspond to the arms of the Y-shaped structure of the antibody, which consist each of the complete light chain paired with the VH and CH1 domains of the heavy chain, and are called the “Fab fragments” (for Fragment antigen binding). Fab fragments were first generated from native immunoglobulin molecules by papain digestion which cleaves the antibody molecule in the hinge region, on the amino-terminal side of the interchains disulfide bonds, thus releasing two identical antigen-binding arms. Other proteases such as pepsin, also cleave the antibody molecule in the hinge region, but on the carboxy-terminal side of the interchains disulfide bonds, releasing fragments consisting of two identical Fab fragments and remaining linked through disulfide bonds; reduction of disulfide bonds in the F(ab′)2 fragments generates Fab′ fragments.

The part of the antigen binding region corresponding to the VH and VL domains is called the Fv fragment (for Fragment variable); it contains the CDRs (complementarity determining regions), which form the antigen-binding site (also termed paratope).

The effector region of the antibody which is responsible of its binding to effector molecules or cells, corresponds to the stem of the Y-shaped structure, and contains the paired CH2 and CH3 domains of the heavy chain (or the CH2, CH3 and CH4 domains, depending on the class of antibody), and is called the Fc region (for Fragment crystallisable region).

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains, which enables the scFv to form the desired structure for antigen binding. The linker is usually rich in glycine for flexibility, as well as serine or threonine for solubility, and can either connect the N-terminus of the VH with the C-terminus of the VL, or vice versa. This scFv fragment retains the specificity of the original antibody, despite removal of the constant regions and the introduction of the linker.

The sequence listing shows the following sequences:
SEQ ID NO: 1 is wild-type human IL2 (253 amino acids, including the signal peptide)
SEQ ID NO: 2 is mature wild-type human IL2 (233 amino acids, without the signal peptide)
SEQ ID NO: 3 is C4BP beta chain (1-252)
SEQ ID NO: 4 is fragment 194-252 of C4BP beta chain
SEQ ID NO: 5 is fragment 137-252 of C4BP beta chain
SEQ ID NO: 6 is the amino acid sequence of human IL-2 fused to the C-terminal region of C4BPß (Hi2cb) including the signal peptide
SEQ ID NO: 7 is the amino acid sequence of mutated IL-2 (N88R) fused to the C-terminal region of C4BPß (Hi2mcb), including the signal peptide
SEQ ID NO: 8 is the GGGGS pattern (linker)
SEQ ID NO: 9 is the amino acid sequence of human IL-2 fused to the C-terminal region of C4BPß (Hi2cb) without the signal peptide
SEQ ID NO: 10 is the amino acid sequence of mutated IL-2 (N88R) fused to the C-terminal region of C4BPß (Hi2mcb) without the signal peptide
SEQ ID NO: 11 is the sequence of E06 scFv VL domain
SEQ ID NO: 12 is the sequence of E06 scFv VH domain
SEQ ID NO: 13 is the sequence of E06 scFv (without HIS tag)
SEQ ID NO: 14 is linker GGGGSGGGGSGGGGS
SEQ ID NO: 15 is the sequence of the targeted fusion protein IL-2-C4bpß-scFvE06 (without peptide signal, and without HIS tag)
SEQ ID NO: 16 is the sequence of the targeted fusion protein IL-2-C4bpß-scFvE06 (with peptide signal, but without HIS tag)
SEQ ID NO: 17 is the sequence of the targeted fusion protein IL-2N88R-C4bpß-scFvE06 (without peptide signal, and without HIS tag)
SEQ ID NO: 18 is the sequence of the targeted fusion protein IL-2N88R-C4bpß-scFvE06 (with peptide signal, but without HIS tag)
SEQ ID NO: 19 is the sequence of the targeted fusion protein IL-2-C4bpß-scFvE06 (with peptide signal, and with HIS tag)
SEQ ID NO: 20 is the sequence of the targeted fusion protein IL-2N88R-C4bpß-scFvE06 (with peptide signal, and with HIS tag)

The chimeric constructs of the invention comprises (i) at least one interleukin 2 moiety; and ii) at least one targeting moiety.

### The IL-2 Moiety

As used herein, Interleukin-2 (IL-2) encompasses mammal wild type Interleukin-2, and variants thereof. Preferably, IL-2 is a human IL-2, or a variant thereof.

Active variants of IL-2 have been disclosed in the literature. Variants of the native IL-2 can be fragments, analogues, and derivatives thereof. By “fragment” is intended a polypeptide comprising only a part of the polypeptide sequence. An “analogue” designates a polypeptide comprising the native polypeptide sequence with one or more amino acid substitutions, insertions, or deletions. Muteins and pseudopeptides are specific examples of analogues. “Derivatives” include any modified native IL-2 polypeptide or fragment or analogue thereof, such as glycosylated, phosphorylated, fused to another polypeptide or molecule, polymerized, etc., or through chemical or enzymatic modification or addition to improve the properties of IL-2 (e.g., stability, specificity, etc.). The IL-2 moiety of active variants generally has at least 75%, preferably at least 80%, 85%, more preferably at least 90% or at least 95% amino acid sequence identity to the amino acid sequence of the reference IL-2 polypeptide, for instance mature wild type human IL-2.

As used herein, “wild type IL-2” means IL-2, whether native or recombinant, comprising the 133 normally occurring amino acid sequence of native human IL-2, whose amino acid sequence is described in Fujita, et. al., PNAS USA, 80, 7437-7441 (1983). SEQ ID NO: 2 (133 amino acids) is the human IL-2 sequence less the signal peptide, consisting of an additional 20 N-terminal amino acids. SEQ ID NO:1 (153 amino acids) is the human IL-2 sequence including the signal peptide.

As used herein, “IL-2 mutein” means a polypeptide in which specific amino acid substitutions to the human mature interleukin-2 protein have been made. All numbering of the amino acids is made with respect to human mature interleukin-2 protein of SEQ ID NO: 2, unless otherwise indicated.

In some embodiments, the cysteine at position 125 is replaced with a neutral amino acid such as serine (C125S), alanine (C125A), threonine (C125T) or valine (C125V).

For example, elimination of the O-glycosylation site results in a more homogenous product when active variant is expressed in mammalian cells such as CHO or HEK cells.

In certain embodiments active variant comprises an additional amino acid mutation which eliminates the O-glycosylation site of IL-2 at a position corresponding to residue 3 of human IL-2. In one embodiment said additional amino acid mutation which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P. In a specific embodiment, said additional amino acid mutation is the amino acid substitution T3A.
Active IL-2 variants that selectively promote T-reg cell proliferation, survival, activation and or function are particularly useful in treating inflammatory and or autoimmune disorders.

By “selectively promote,” it is meant that the active variant promotes the activity in T-reg cells but has limited or lacks the ability to promote the activity in non-regulatory T cells. Further described herein are assays to screen for active variants that selectively promote T-reg cell proliferation, survival, activation and/or function. Methods for determining whether a variant IL-2 polypeptide is active are available in the art. See e.g. WO2016/014428. An active variant is defined as a variant that shows an ability to stimulate Tregs, including variants with an improved ability, or a similar ability, or even a reduced ability to stimulate Tregs when compared to wild-type IL-2 or aldesleukin (as defined below), to the extent it does not stimulate Teffs more than it stimulates Tregs. Methods for testing whether a candidate molecule stimulate T cells, Tregs in particular, or NK cells are well-known. Variants may be tested for their ability to stimulate effector T cells (such as CD8+ T cells), CD4+Foxp3+ Tregs, or NK cells. In a preferred embodiment, the active variant shows a reduced ability to stimulate NK cells, compared to wild type IL2 or aldesleukin. Monitoring STAT5 phosphorylation is a simple way of assessing variants for their ability to preferentially stimulate Tregs over Teff, as described in Yu et al, Diabetes 2015; 64:2172-2183. In a particular embodiment, a variant is particularly useful when a given level of STAT5 phosphorylation is achieved with doses at least 10 times inferior for Tregs than for other immune cells, including Teffs.

Said active variants induce signaling events that preferentially induce survival, proliferation, activation and/or function of Treg cells. In certain embodiments, the IL-2 variant retains the capacity to stimulate, in Treg cells, STAT5 phosphorylation and/or phosphorylation of one or more of signaling molecules downstream of the IL-2R, e.g., p38, ERK, SYK and LCK. In other embodiments, the IL-2 variant retains the capacity to stimulate, in Treg cells, transcription or protein expression of genes or proteins, such as FOXP3, Bcl-2, CD25 or IL-10, that are important for Treg cell survival, proliferation, activation and/or function. In other embodiments, the IL-2 variant exhibits a reduced capacity to stimulate endocytosis of IL-2/IL-2R complexes on the surface of CD25+ T cells. In other embodiments, the IL-2 variant demonstrates inefficient, reduced, or absence of stimulation of PI3-kinase signaling, such as inefficient, reduced or absent phosphorylation of AKT and/or mTOR (mammalian target of rapamycin). In yet other embodiments, the IL-2 variant retains the ability of wild type IL-2 to stimulate STAT5 phosphorylation and/or phosphorylation of one or more of signaling molecules downstream of the IL-2R in Treg cells, yet demonstrates inefficient, reduced, or absent phosphorylation of STAT5, AKT and/or mTOR or other signaling molecules downstream of the IL-2R in FOXP3−CD4+ or CD8+ T cells or NK cells. In other embodiments, the IL-2 variant is inefficient or incapable of stimulating survival, growth, activation and/or function of FOXP3-CD4+ or CD8+ T cells or NK cells.

In all cases, these variants have the capacity to stimulate cell lines such as CTLL-2 or HT-2 which can be universally used to determine their biological activity. For instance, the biological activity of IL-2 may be determined by a cell-based assay performed on HT-2 cell line (clone A5E, ATCC® CRL-1841™) whose growth is dependent on IL-2. Cell growth in the presence of a range of test interleukin-2 product is compared with the growth recorded with IL-2 international standard (WHO 2nd International Standard for INTERLEUKIN 2 (Human, rDNA derived) NIBSC code: 86/500). Cell growth is measured after addition and transformation of [3-(4,5-dimethylthiazol-2-yl)-5-(3-carboxymethoxyphenyl)-2-(4-sulfophenyl)-2H-tetrazolium (inner salt, MTS) into formazan by active viable cells. Formazan concentration is then measured by spectrophotometry at 490 nm.

Examples of IL-2 variants are disclosed, for instance, in EP109748, EP136489, U.S. Pat. No. 4,752,585; EP200280, EP118617, WO99/60128, EP2288372, U.S. Pat. Nos. 9,616,105, 9,580,486, WO2010/085495, WO2016/164937.

For instance, certain mutations may result in a reduced affinity for the signaling chains of the IL-2 receptor (IL-2Rβ/CD122 and/or IL-2Rγ/CD132) and/or a reduced capacity to induce a signaling event from one or both subunits of the IL-2 receptor. Other mutations may confer higher affinity for CD25 (IL-2Rα). In both cases, those mutations define active variants that preferentially induce survival, proliferation, activation and/or function of Treg. This property may be monitored using surface plasmon resonance.

Particular examples of useful variants include IL-2 muteins which show at least one amino acid substitution at position D20, N30, Y31, K35, V69, Q74, N88, V91, or Q126, numbered in accordance with wild type IL-2, meaning that the chosen amino acid is identified with reference to the position at which that amino acid normally occurs in the mature sequence of wild type IL-2 of SEQ ID NO:2.

Preferred IL-2 muteins comprise at least one substitution at position D20H, D20I, D20Y, N30S, Y31H, K35R, V69AP, Q74, N88R, N88D, N88G, N88I, V91K, or Q126L.

In some embodiments, the IL-2 mutein molecule comprises a V91K substitution. In some embodiments, the IL-2 mutein molecule comprises a N88D substitution. In some embodiments, the IL-2 mutein molecule comprises a N88R substitution. In some embodiments, the IL-2 mutein molecule comprises a substitution of H16E, D84K, V91N, N88D, V91K, or V91R, any combinations thereof. In some embodiments, these IL-2 mutein molecules also comprise a substitution at position 125 as described herein. In some embodiments, the IL-2 mutein molecule comprises one or more substitutions selected from the group consisting of: T3N, T3A, L12G, L12K, L12Q, L 12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D20I, D20Y, D20F, D20G, D20T, D20W, M23R, R81A, R81G, R81 S, R81T, D84A, D84E, D84G, D84I, D84M, D84Q D84R, D84S, D84T, S87R, N88A, N88D, N88E, N88I, N88F, N88G, N88M, N88R, N88S, N88V, N88W, V91D, V91E, V91G, V91 S, 192K, 192R, E95G, and Q126. In some embodiments, the amino acid sequence of the IL-2 mutein molecule differs from the amino acid sequence set forth in mature IL-2 sequence with a C125A or C125S substitution and with one substitution selected from T3N, T3A, L12G, L12K, L12Q L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81 S, R81T, D84A, D84E, D84G, D84I, D84M, D84Q, D84R, D84S, D84T, S87R, N88A, N88D, N88E, N88F, N88I, N88G, N88M, N88R, N88S, N88V, N88W, V91D, V91E, V91G, V91 S, 192K, 192R, E95G, Q126I, Q126L, and Q126F. In some embodiments, the IL-2 mutein molecule differs from the amino acid sequence set forth in mature IL-2 sequence with a C125A or C125S substitution and with one substitution selected from D20H, D20I, D20Y, D20E, D20G, D20W, D84A, D84S, H16D, H16G, H16K, H16R, H16T, H16V, 192K, 192R, L12K, L19D, L19N, L19T, N88D, N88R, N88S, V91D, V91G, V91K, and V91S. In some embodiments, the IL-2 mutein comprises N88R and/or D20H mutations.

These substitutions can be used alone or in combination with one another. In some embodiments, the mutein comprises each of these substitutions. In some embodiments, the mutein comprises 1, 2, 3, 4, 5, 6, 7, or 8 of these mutations.

In some embodiments, the IL-2 mutein comprises a N88R or a N88D mutation, preferably N88R. In some embodiments, the IL-2 mutein comprises a C125A or C125S mutation. These substitutions can be used alone or in combination with one another. In some embodiments, the mutein comprises 1, 2, 3, 4, 5, 6, 7, or 8 of these mutations. In some embodiments, the mutein comprises each of these substitutions.

In a particular embodiment, the IL-2 moiety is aldesleukin. Aldesleukin is the active ingredient of Proleukin®. Aldesleukin is a variant of mature human IL-2 comprising two amino acid modifications as compared to the sequence of mature human IL-2 (SEQ ID NO:2): the deletion of the first amino acid (alanine) and the substitution of cysteine at position 125 by serine. Conservative modifications and substitutions at other positions of IL-2 (i. e., those that have a minimal effect on the secondary or tertiary structure of the mutein) are encompassed. Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: -ala, pro, gly, gln, asn, ser, thr; -cys, ser, tyr, thr; -val, ile, leu, met, ala, phe; -lys, arg, his; -phe, tyr, trp, his; and -asp, glu.

Variants with mutations which disrupt the binding to the α subunit of IL-2R are not preferred, as those mutants may have a reduced capacity to stimulate Tregs.
Active IL-2 variants that promote Teff cell proliferation, survival, activation and or function may be useful in treating cancers.

Such active variants of IL-2 comprise at least one amino acid mutation that abolishes or reduces affinity of the mutant IL-2 polypeptide to the α-subunit of the IL-2 receptor (CD25) and preserves affinity of the mutant IL-2 polypeptide to the intermediate-affinity IL-2 receptor, each compared to a wild-type IL-2 polypeptide. This property may be monitored using surface plasmon resonance.

Preferred active variants include IL-2 mutein comprising F42A, K43N, Y45A, and/or E62A substitution(s).

Active variants such as mutants of human IL-2 (hIL-2) with decreased affinity to CD25 may for example be generated by amino acid substitution at amino acid position 35, 38, 42, 43, 45, 62 or 72 or combinations thereof (numbering relative to the human IL-2 sequence SEQ ID NO: 2). Exemplary amino acid substitutions include K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, Y45K, E62G, E62A, E62S, E62T, E62Q, E62E, E62N, E62D, E62R, E62K, L72G, L72A, L72S, L72T, L72Q, L72E, L72N, L72D, L72R, and L72K. Particular active variants useful in the chimeric construct for the present invention comprise an amino acid mutation at an amino acid position corresponding to residue 42, 45, or 72 of human IL-2, or a combination thereof. In one embodiment said amino acid mutation is an amino acid substitution selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, more specifically an amino acid substitution selected from the group of F42A, Y45A and L72G. These active variants exhibit substantially similar binding affinity to the intermediate-affinity IL-2 receptor, and have substantially reduced affinity to the α-subunit of the IL-2 receptor and the high-affinity IL-2 receptor (IL2Rαβγ) compared to a wild-type form of the IL-2 mutant.

Other characteristics of useful active variants may include the ability to induce proliferation of IL-2 receptor-bearing T and/or NK cells, the ability to induce IL-2 signaling in IL-2 receptor-bearing T and/or NK cells, the ability to generate interferon (IFN)-y as a secondary cytokine by NK cells, a reduced ability to induce elaboration of secondary cytokines—particularly IL-10 and TNF-α—by peripheral blood mononuclear cells (PBMCs), a reduced ability to activate regulatory T cells, a reduced ability to induce apoptosis in T cells, and a reduced toxicity profile in vivo.

Particular active variants comprise three amino acid mutations that abolish or reduce affinity of the active variants to the α-subunit of the IL-2 receptor but preserve affinity of the active variant to the intermediate affinity IL-2 receptor. In one embodiment said three amino acid mutations are at positions corresponding to residue 42, 45 and 72 of human IL-2. In one embodiment said three amino acid mutations are amino acid substitutions. In one embodiment said three amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In a specific embodiment said three amino acid mutations are amino acid substitutions F42A, Y45A and L72G (numbering relative to the human IL-2 sequence of SEQ ID NO: 2). In certain embodiments said amino acid mutation reduces the affinity of the mutant IL-2 polypeptide to the α-subunit of the IL-2 receptor by at least 5-fold, specifically at least 10-fold, more specifically at least 25-fold. In embodiments where there is more than one amino acid mutation that reduces the affinity of the active variant to the α-subunit of the IL-2 receptor, the combination of these amino acid mutations may reduce the affinity of the active variant to the α-subunit of the IL-2 receptor by at least 30-fold, at least 50-fold, or even at least 100-fold. In one embodiment said amino acid mutation or combination of amino acid mutations abolishes the affinity of the active variant to the α-subunit of the IL-2 receptor so that no binding is detectable by surface plasmon resonance.

Substantially similar binding to the intermediate-affinity receptor, i.e. preservation of the affinity of the mutant IL-2 polypeptide to said receptor, is achieved when the active variant exhibits greater than about 70 percent of the affinity of a wild-type form of the IL-2 mutant to the intermediate-affinity IL-2 receptor. Active variants useful in the invention may exhibit greater than about 80 percent and even greater than about 90 percent of such affinity.

Reduction of the affinity of IL-2 for the α-subunit of the IL-2 receptor in combination with elimination of the O-glycosylation of IL-2 results in an IL-2 protein with improved properties. In a specific embodiment, the active variant can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, cytotoxic activity in a NK cell, differentiation in a NK cell, cytokine secretion by an activated T cell or an NK cell, and NK/lymphocyte activated killer (LAK) antitumor cytotoxicity.

In some embodiments, these active variants also comprise a substitution at position 125 as described herein.

### The Targeting Moiety

The chimeric construct further comprises a targeting moiety, which is able to target IL-2 to inflammatory tissues.

In particular, the targeting moiety binds to an oxidized protein or oxidized lipid. In the context of the present invention, the oxidized protein or oxidized lipid is found in inflammatory tissues. In a particular embodiment, the targeting moiety binds to an oxidized protein or oxidized lipid, which contributes to inflammation or which is involved in the inflammatory response. In a particular embodiment, the targeting moiety binds to an oxidized protein or oxidized lipid, which is induced by and/or contribute to oxidative damage and inflammation.

In a particular embodiment, the targeting moiety binds to a pro-inflammatory oxidized protein or oxidized lipid. By “pro-inflammatory” is meant an oxidized protein or lipid that is a positive mediator of inflammation. For example, a pro-inflammatory protein or lipid may induce the secretion of inflammatory cytokine(s) and/or the recruitment of effector cells such as monocytes and macrophages. An inflammatory cytokine is a type of signaling molecule that is secreted from immune cells like helper T cells (Th) and macrophages, and certain other cell types that promote inflammation. They include interleukin-1 (IL-1), IL-12, and IL-18, tumor necrosis factor alpha (TNF-α), interferon gamma (IFNγ), or granulocyte-macrophage colony stimulating factor (GM-CSF).

In a particular embodiment, the oxidized protein or oxidized lipid is involved or trigger sterile inflammation. “Sterile inflammation” is an inflammation which occurs in the absence of any microorganisms, and that is elicited in response to damage-associated molecular patterns (DAMPs), which are released locally in response to tissue damage. DAMPs are intracellular and extracellular host-derived molecules that are not usually sensed by the immune system but that are released or become modified into altered self-molecules upon tissue damage. In analogy to pathogen-induced inflammation, sterile inflammation is triggered by the activation of the innate immune response through the recognition of DAMPs by pattern recognition receptors (PRRs), resulting in the enhanced secretion of cytokines and chemokines. Membrane-bound PRRs, such as Toll-like receptors (TLRs), and intracellular PRRs, such as the inflammasome, are key mediators of sterile inflammation. Cytokines belonging to the interleukin-1 (IL-1) family have been proposed to be important drivers of sterile inflammation. Increased cytokine and chemokine secretion at the site of initial damage ultimately results in an enhanced recruitment of immune cells, such as neutrophils and macrophages. The resolution of sterile inflammation should lead to tissue repair and the re-establishment of homeostasis. Unresolved sterile inflammation is implicated in the development of several medical conditions, such as autoimmune diseases, gout, Alzheimer disease, and atherosclerosis.

In a particular embodiment, the targeting moiety binds to an “oxidation-specific epitope” (OSE). Said oxidation-specific epitope (OSE) is present on the oxidized protein or the oxidized lipid, as defined above.

Cells produce reactive oxygen species (ROS) whose biological effects depend on the amount produced. At low concentrations, ROS are involved in proliferation, differentiation and cell metabolism while at high concentrations, they are involved in the formation of neutrophil extracellular traps (NETs) that promote microbial elimination. In response to certain exogenous and endogenous stimuli, such as inflammation, this balance can be disrupted and lead to the accumulation of ROS that participate in oxidative stress causing irreversible alteration of DNA, RNA, proteins and lipids. A major consequence of oxidative stress is lipid peroxidation, which generates a number of highly reactive breakdown products, that in turn react with lipids, apoproteins and proteins, thereby forming stable covalent adduct and creating “oxidation-specific epitopes” (OSE). OSE can be recognized as isolated lipids or covalently associated with a protein. Examples of OSE include oxidized phospholipids (OxPL). See e.g. Binder et al, (2016) Nat Rev Immunol, 16(8):485-97, for a review and examples. OSEs can be generated by the modification of proteins with truncated phospholipids, such as oxidized phosphatidylcholine, oxidized cardiolipin (OxCL), oxidized phosphatidylserine (OxPS) and oxidized phosphatidylethanolamine (OxPE). Other examples of OSEs include malondialdehyde (MDA) epitopes, 2-(ω-carboxyethyl) pyrrole (CEP) epitopes and 4-hydroxynonenal (4-HNE) epitopes.

OSE have been documented in oxidized lipoproteins and on the surface of dying cells and circulating microparticles, and their ability to trigger robust pro-inflammatory responses has been demonstrated (Tsiantoulas et al. (2015). Circulating microparticles carry oxidation-specific epitopes and are recognized by natural IgM antibodies 1. J. Lipid Res. 56, 440-448). In particular, the recognition by PRRs (“Pattern Recognition Receptor”) of the OSE, considered as PAMPs (“Pathogen-Associated Molecular Patterns”), will induce the expression of pro-inflammatory cytokines and the activation of cellular effectors such as monocytes and macrophages (Miller et al. (2011). Oxidation-Specific Epitopes are Danger Associated Molecular Patterns Recognized by Pattern Recognition Receptors of Innate Immunity. Circ. Res. 108, 235-248.).

Due to their accumulation in inflammatory conditions, OSE-modified proteins or lipids may be used as a target, for targeting the IL-2 moiety to inflammatory tissues.

In a particular embodiment, the targeting moiety binds to an OSE, wherein the OSE is: a malondialdehyde (MDA) epitope; a 2-(ω-carboxyethyl) pyrrole (CEP) epitope; a 4-hydroxynonenal (4-HNE) epitope, an oxidized phospholipid (OxPL), a phosphocholine-containing oxidized phospholipid (PC-OxPL), an oxidized phosphatidylethanolamine (OxPE), an oxidized phosphatidylserine (OxPS) or an oxidized cardiolipin (OxCL).

In a preferred embodiment, the targeting moiety binds to an oxidized phospholipid (OxPL).

Preferably, the targeting moiety binds to a phosphocholine-containing oxidized phospholipid (PC-OxPL). Oxidized phospholipids with a phosphocholine headgroup were shown to be highly pro-inflammatory and proatherogenic and are both induced by and propagate oxidative damage and inflammation. They are present in a wide spectrum of inflammatory diseases, including atherosclerosis, rheumatoid arthritis, diabetic nephropathy, CNS diseases including multiple sclerosis, and a spectrum of acute and chronic pulmonary diseases.

In a particular embodiment, the targeting moiety is an antibody or an antibody fragment, such as a Fab, Fab′, F(ab′)2, Fv or scFv fragment.

In a preferred embodiment, the targeting moiety is a scFv fragment. Indeed, a scFv fragment lacks a Fc domain and therefore has a silenced effector function.

In a particular embodiment, the targeting moiety is selected from the group consisting of: a E06 antibody or a E06 antibody fragment such as a E06 scFv; a LR04 antibody or a LR04 antibody fragment such as a LR04 scFv; a NA17 antibody or a NA17 antibody fragment such as a NA17 scFv; a E014 antibody or a E014 antibody fragment such as a E014 scFv; a MDA2 antibody or a MDA2 antibody fragment such as a MDA2 scFv; a IK17 antibody or a IK17 antibody fragment such as a IK17 scFv; and a LR01 antibody or a LR01 antibody fragment such as LR01 scFv.

In a preferred embodiment, the targeting moiety is a E06 antibody or a E06 antibody fragment such as a Fab, Fab′, F(ab′)2, Fv or scFv fragment of E06 antibody, or functional variants thereof. E06 is a natural IgM autoantibody cloned from apolipoprotein E-deficient mice (apoE−/−) that binds to the phosphocholine (PC) head group of oxidized but not normal phospholipids (Friedman et al. (2001) Correlation of antiphospholipid antibody recognition with the structure of synthetic oxidized phospholipids: Importance of Schiff base formation and Aldol condensation. J Biol Chem.; 277:7010-7020). Interestingly, E06 is structurally and functionally identical to classic “natural” murine T15 anti-PC antibodies that are of B-1 cell origin and are reported to provide optimal protection from virulent pneumococcal infection.

In a particular embodiment, the targeting moiety is a E06 scFv or a functional variant thereof.

The term “functional variant” or “derivative”, designates a sequence that differs from the parent sequence to which it refers by deletion, substitution or insertion of one or several amino acids, without substantially impacting the function of the antibody or the fragment thereof. Preferably, the functional variant shows 75%, 80%, 85%, 90%, 95%, 96%, 97%, 98% or at least 99% identity with the native sequence. A functional variant of an antibody or a fragment thereof possesses similar antigen-binding affinity relative to the reference antibodies (e.g., having a KD less than 1×10-7 M, 10-8 M, preferably less than 1×10-9 or 1×10-10 M). The affinity of the binding is defined by the terms ka (associate rate constant), kd (dissociation rate constant), or KD (equilibrium dissociation). Typically, specifically binding when used with respect to an antibody refers to an antibody that specifically binds to (“recognizes”) its target(s) with an affinity (KD) value less than 10-7 M, preferably less than 10-8 M, e.g., less than 10-9 M or 10-10 M. A lower KD value represents a higher binding affinity (i.e., stronger binding) so that a KD value of 10-9 indicates a higher binding affinity than a KD value of 10-8.

In a particular embodiment, the E06 scFv comprises:
a variable heavy chain (VH) domain comprising or consisting of an amino acid sequence as shown in SEQ ID NO:12, or an amino acid sequence having at least 75%, 80%, 85%, 90%, 95%, 96%, 97%, 98% or at least 99% identity with SEQ ID NO:12; and
a variable light chain (VL) domain comprising or consisting of an amino acid sequence as shown in SEQ ID NO:11, or an amino acid sequence having at least 75%, 80%, 85%, 90%, 95%, 96%, 97%, 98% or at least 99% identity with SEQ ID NO:11.

In a particular embodiment, the VH domain and the VL domain of the E06 scFv are fused through an amino acid linker. The term “linker” refers to a (poly) peptide comprising 5 to 80 amino acids, preferably 5 to 30, still preferably 10 to 20 amino acids. Suitable linkers are known in the art. In some embodiments, the linker comprises GGGGS (SEQ ID NO: 8) repeats. Linkers composed of small, non-polar (e.g. Gly) or polar (e.g. Ser or Thr) amino acids provide flexibility, and allows for mobility of the connecting functional domains. In a preferred embodiment, the linker is the linker of SEQ ID NO: 14 or a linker having an amino acid sequence having at least 75%, 80%, 85%, 90%, 95%, 96%, 97%, 98% or at least 99% identity with SEQ ID NO: 14.

In a particular embodiment, the E06 scFv fragment comprises of consists of the amino acid sequence as shown in SEQ ID NO: 13 or an amino acid sequence having at least 75%, 80%, 85%, 90%, 95%, 96%, 97%, 98% or at least 99% identity with SEQ ID NO: 13.

In another particular embodiment, the targeting moiety is a LR04 antibody or a LR04 antibody fragment such as a Fab, Fab′, F(ab′)2, Fv or scFv fragment of LR04 antibody, or functional variants thereof. LR04 is a monoclonal IgM antibody against MDA epitopes cloned from murine Ldlr−/− spleens on atherogenic diet (Amir et al. Peptide mimotopes of malondialdehyde epitopes for clinical applications in cardiovascular disease. J Lipid Res. 2012; 53:1316-1326).

In another particular embodiment, the targeting moiety is a NA17 antibody or a NA17 antibody fragment such as a Fab, Fab′, F(ab′)2, Fv or scFv fragment of NA17 antibody, or functional variants thereof. NA17 is a MDA-specific natural mAb, cloned from the spleen of a B-1 cell reconstituted Rag1−/− mice (Chou et al. Oxidation-specific epitopes are dominant targets of innate natural antibodies in mice and humans. J Clin Invest. 2009; 119 (5): 1335-1349).

In another particular embodiment, the targeting moiety is a E014 antibody or a E014 antibody fragment such as a Fab, Fab′, F(ab′)2, Fv or scFv fragment of E014 antibody, or functional variants thereof. E014 is a monoclonal IgM NAb cloned from the spleens of atherosclerotic Apoe−/− mice, that has been shown to bind MDA-epitopes (Palinski et al. (1996). Cloning of monoclonal autoantibodies to epitopes of oxidized lipoproteins from apolipoprotein E-deficient mice. Demonstration of epitopes of oxidized low density lipoprotein in human plasma. J. Clin. Invest. 98, 800-814).

In another particular embodiment, the targeting moiety is a MDA2 antibody or a MDA2 antibody fragment such as a Fab, Fab′, F(ab′)2, Fv or scFv fragment of MDA2 antibody, or functional variants thereof. MDA2 is a murine monoclonal IgG type antibody specific for MDA-lysine epitopes. It specifically binds MDA-LDL, and other MDA-modified proteins (Rosenfeld et al (1990). Distribution of oxidation specific lipid-protein adducts and apolipoprotein B in atherosclerotic lesions of varying severity from WHHL rabbits. Arteriosclerosis; 10:336-349).

In another particular embodiment, the targeting moiety is a IK17 antibody or a IK17 antibody fragment such as a Fab, Fab′, F(ab′)2, Fv or scFv fragment of IK17 antibody, or functional variants thereof. IK17 is a human monoclonal IgG antibody fragment binding to MDA-LDL and copper OxLDL (Shaw et al. (2001) Human-derived anti-oxidized LDL autoantibody blocks uptake of oxidized LDL by macrophages and localizes to atherosclerotic lesions in vivo. Arterioscler Thromb Vasc Biol.; 21:1333-1339). IK17 was isolated from a phage display library from a patient with coronary artery disease with high plasma autoantibody titers to MDA-LDL. Because IK17 is a human autoantibody it has potential advantages over murine antibodies, including improved pharmacokinetics and reduced immunologic reactions.

In another particular embodiment, the targeting moiety is a LR01 antibody or a LR01 antibody fragment such as a Fab, Fab′, F(ab′)2, Fv or scFv fragment of LR01 antibody, or functional variants thereof. LR01 is a germline-encoded NAb isolated from the spleens of atherosclerotic Ldlr−/− mice. LR01 was found to be directed against oxidized but not native cardiolipin (Tuominen et al. (2006). A natural antibody to oxidized cardiolipin binds to oxidized low-density lipoprotein, apoptotic cells, and atherosclerotic lesions. Arterioscler. Thromb. Vasc. Biol. 26, 2096-2102).

### Optional Moiety Having Dimerization Properties

The chimeric construct of the invention, which comprises a IL-2 moiety and a targeting moiety, may optionally comprise a moiety having multimerization properties, i.e. a fragment or moiety that is able to form multimeric proteins.

Preferably, the chimeric construct of the invention, which comprises a IL-2 moiety and a targeting moiety, may optionally comprise a moiety having dimerization properties, i.e. a fragment or moiety that is able to form dimeric proteins.

For example, the chimeric construct may further comprise a Fc fragment of an IgG, or a functional variant thereof which has the capacity to form at least one dimer, for example a homodimer or a heterodimer, a trimer, a tetramer or any multimer containing a different number of chimeric constructs.

In a particular embodiment, the chimeric construct of the invention, which comprises a IL-2 moiety and a targeting moiety, may optionally comprise a beta chain of the C4b-binding protein (C4BPβ) or at least one fragment or functional variant thereof that is capable of forming a dimeric protein.

### The C4BPβ or C4BPβ Fragment

The C4BP protein is involved in coagulation and the complement system. The major form of C4BP is composed of 7 identical 75 kD alpha chains and one 45 kD beta chain. The alpha and beta chains respectively contain 8 and 3 SCR (short consensus repeat) domains, those motifs being found in many complement-regulating proteins and constituted by 50-70 amino acids organized into beta sheets. The amino acid sequence of the beta chain of human C4BP is shown as SEQ ID NO:3.

A nucleic acid sequence corresponding to this polypeptide sequence has also been described by Hillarp and Dahlback (1990, PNAS, vol 87, pp 1183-1187). The role of the alpha chain in polymerizing the C4BP protein has been studied by Kask et al (Biochemistry 2002, 41, 9349-9357). Those authors have shown that the C-terminal portion of the alpha chain, in particular its helical structure and the presence of two cysteines, is necessary for polymerization of the C4BP protein when the alpha chain is expressed in a heterologous system.

European patent application 2 227 030 describes the production of heteromultimeric recombinant proteins by using C-terminal fragments of the alpha and beta chains of the C4BP protein in fusion with polypeptides of interest. U.S. Pat. No. 7,884,190 describes the use of the beta chain of the C4BP protein, independently of its use in association with the alpha chain of the C4BP protein for the production of dimeric proteins.

The C4BP protein used to carry out the invention is advantageously the human C4BP protein. In a preferred embodiment, the chimeric construct comprises a fragment of the C4BPβ chain that comprises or consists of at least amino acids 194 to 252 (SEQ ID NO: 4).

Sequences coding for longer fragments of the beta chain, or even the whole beta chain, may also be used. For certain applications, it is preferable to avoid using a sequence coding for a beta chain which is capable of binding the S protein participating in coagulation. If the selected sequence codes for a fragment containing the two first SCR motifs of the beta chain, these will preferably by versions mutated by addition, deletion or substitution of amino acids to cut out with the possibility of interaction with the S protein. SCR motifs and/or [GS] domains may be added with the aim of modifying, for example increasing, the flexibility of the fusion polypeptide obtained or to allow the chimeric protein to adopt a suitable conformation to form multimers, particularly dimers.

A longer fragment of C4BPβ that extends at the N-term up to at most amino acid 135 may be used.

In a particular embodiment, the fragment of the C4BPβ chain may comprise or consist of at least amino acids 185 to 252, 180 to 252, 175 to 252, 170 to 252, 165 to 252, 160 to 252, 155 to 252, 150 to 252, 145 to 252, 140 to 252, or 135 to 252 (with respect to SEQ ID NO:3).

In a particular embodiment, the fragment of the C4BPβ chain comprises or consists of at least amino acids 137 to 252 (SEQ ID NO: 5).

A functional variant of C4BPβ may be used. The functional variant has maintained the capacity to form at least one dimer, for example a homodimer or a heterodimer, a trimer, a tetramer or any multimer containing a different number of chimeric proteins.

Within the context of the invention, the term “functional variant of a fragment of the C4BPβ chain” means a polypeptide sequence modified with respect to the sequence of fragment of the beta chain by deletion, substitution or addition of one or more amino acids, said modified sequence retaining, however, the capacity to form at least dimer proteins using the method of the invention. More precisely, the production of dimer proteins using a sequence coding for a functional variant of the fragment may be at least 80% equal to that obtained with a native sequence coding for the fragment (SEQ ID NO: 3, or a fragment thereof), preferably at least 90%, still preferably 95%) in an identical expression system. Preferably, the variant is such that more than 80% of the fusion polypeptides which it contains are produced in the form of dimers in a eukaryotic expression system in accordance with the invention.

In a particular embodiment, a variant of the fragment of the beta chain is encoded by a nucleic acid that is capable of hybridizing under stringent conditions with the wildtype sequence coding for the fragment, as described by Hillarp and Dahlback (1990, PNAS, Vol. 87, pp 1183-1187). The term “stringent conditions” means conditions which allow specific hybridization of two single strand DNA sequences at about 65° C., for example, in a solution of 6*SSC, 0.5% SDS, 5* Denhardt's solution and 100 μg of non specific DNA or any other solution with an equivalent ionic strength and after washing at 65° C., for example in a solution of at most 0.2*SSC and 0.1% SDS or any other solution with an equivalent ionic strength.

Preferably, the nucleotide sequence coding for a functional variant of said wildtype fragment and hybridizing under stringent conditions with the sequence coding for said fragment has, in the portion which hybridizations, a length of at least 50%, preferably at least 80%, of the length of the sequence coding for the fragment. In a particular implementation, the nucleotide sequence coding for a functional variant of said fragment and hybridizing under stringent conditions with the sequence coding for said fragment has, in the portion which hybridizations, substantially the same length as the sequence coding for said fragment.

In a further implementation, a functional variant is a modified sequence of the wildtype fragment one or more amino acids of which, not essential to the dimerization function, have been removed or substituted and/or one or more amino acids essential to dimerization have been replaced by amino acids with equivalent functional groups (conservative substitution). It is particularly recommended that the two cysteines, located at positions 201 and 215, and the peptide structure around these cysteines be conserved to allow the formation of disulfide bridges which are necessary for dimerization, for example by conservation of at least 3 amino acids upstream and downstream of each cysteine. In particular, a functional variant may also be obtained by inserting a heterologous sequence of the beta chain, and in particular domains of the alpha chain of C4BP, between the cysteines responsible for dimerization or, in contrast, by doing away with certain amino acids present between those same cysteines. Alternatively, a functional variant may be produced by point modification of certain amino acids, in particular substitution of a cysteine responsible for dimerization by a neutral amino acid as regards implication in the dimerization process (for example the amino acids A, V, F, P, M, I, L and W) and at the same time substituting another amino acid by a cysteine to conserve the capacity to form intracatenary and/or intercatenary disulfide bridges between the cysteines. These modifications thus result in a variation in the distance between the various cysteines involved in the multimerization process, in particular dimerization.

Preferably, less than 50% of the amino acids of the 194 to 252 fragment are done away with or replaced, preferably less than 25% or even less than 10% (for example 5 amino acids or fewer) or less than 5% (e.g. 1 or 2 amino acids).

In a particular embodiment, the functional variant comprises or consists of
a) a modified sequence of the fragment (preferably the 194-252 fragment) of C4BPβ, wherein less than 25 percent of the amino acids of the fragment (preferably the 194-252 fragment), preferably less than 10 percent, have been cut out or replaced, in which the cysteines located in positions 202 and 216 (numbered with respect to SEQ ID NO: 3) as well as at least 3 amino acids upstream and downstream of each cysteine have been conserved; or
b) a modified sequence of the fragment (preferably the 194-252 fragment) of the C4BPβ, wherein a cysteine responsible for dimerization is substituted with an amino acid, preferably selected from alanine, valine, phenylalanine, proline, methionine, isoleucine, leucine and tryptophan, and another amino acid of the fragment is substituted with a cysteine; or
c) a sequence of the fragment (preferably the 194-252 fragment) of C4BPβ modified by insertion of a sequence which is heterologous to the beta chain, between the cysteines responsible for dimerization; or
d) a sequence of the fragment (preferably the 194-252 fragment) of C4BPβ modified by cutting out amino acids between the cysteines responsible for dimerization.

### The Chimeric Constructs

Preferably, the IL2 moiety and the targeting moiety of the chimeric construct are fused to each other. The IL2 moiety may be fused to the N-terminus or to the C-terminus of the targeting moiety. In a preferred embodiment, the IL-2 moiety is fused to the N-terminus of the targeting moiety. Preferably, the C-terminus of the IL-2 moiety is fused to the N-terminus of the targeting moiety.

The IL2 moiety and the targeting moiety may be fused in frame (directly) or through an amino acid linker, preferably a polyG linker. In the context of the present invention, the term “linker” refers to a (poly) peptide comprising 5 to 80 amino acids, preferably 5 to 30, still preferably 10 to 20 amino acids. Suitable linkers are known in the art. In some embodiments, the linker comprises GGGGS (SEQ ID NO: 8) repeats, although an artisan skilled in the art will recognize that other sequences following the general recommendations (Argos, 1990, J Mol Biol. 20; 211(4):943-58; George R, Heringa J. An analysis of protein domain linkers: their classification and role in protein folding. Protein Eng. 2002; 15:871-879) can also be used. Linkers composed of small, non-polar (e.g. Gly) or polar (e.g. Ser or Thr) amino acids provide flexibility, and allows for mobility of the connecting functional domains. In a preferred embodiment, the IL2 moiety and the targeting moiety are liked through the linker of SEQ ID NO: 14 or a linker having an amino acid sequence having at least 75%, 80%, 85%, 90%, 95%, 96%, 97%, 98% or at least 99% identity with SEQ ID NO: 14.

In a particular embodiment, the chimeric construct further comprises a moiety having dimerization properties, such as a of C4BPβ or a functional fragment thereof, as described above. Such chimeric construct preferably forms a homodimer, or may be used to produce a heterodimer, as described below. In a particular embodiment, (i) the IL2 moiety, (ii) the targeting moiety and (iii) the moiety having dimerization properties are fused to each other, in frame (directly) or through amino acid linker(s).

In a preferred embodiment the IL-2 moiety is fused at the N-terminus of C4BPβ or said functional fragment thereof. Preferably, the IL-2 moiety is fused at the N-terminus of C4BPβ or said functional fragment thereof, through an amino acid linker, preferably a polyG linker. In some embodiments, the IL-2 moiety is fused to the C4BPβ moiety via a linker comprising GGGGS (SEQ ID NO: 8) repeats, preferably via the linker of SEQ ID NO: 14 or a linker having an amino acid sequence having at least 75%, 80%, 85%, 90%, 95%, 96%, 97%, 98% or at least 99% identity with SEQ ID NO: 14.

In a particular embodiment, the amino acid sequence corresponding to the IL-2 moiety fused to the C4BPβ moiety (“IL2-C4BPβ”), comprises or consists of SEQ ID NO:9 or SEQ ID NO:10.

In a preferred embodiment the C-terminus of the IL-2 moiety is fused at the N-terminus of C4BPβ or said functional fragment thereof, wherein the C-terminus of C4BPβ or said fragment thereof is linked to the N-terminus of the targeting moiety.

In a particular embodiment, the chimeric construct comprises from N-terminus to C-terminus:
the IL-2 moiety
optionally a linker; and
the targeting moiety.

In a preferred embodiment, the chimeric construct comprises from N-terminus to C-terminus:
the IL-2 moiety
optionally a linker;
the moiety having dimerization properties, such as a of C4BPβ or a functional fragment thereof;
optionally a linker; and
the targeting moiety.

In a preferred embodiment, the chimeric construct comprises or consists of the amino acid sequence of SEQ ID NO:15 to 20, or comprises or consists of an amino acid sequence having at least 75%, 80%, 85%, 90%, 95%, 96%, 97%, 98% or at least 99% identity with SEQ ID NO: 15 to 20. In a preferred embodiment, the chimeric construct comprises or consists of the amino acid sequence of SEQ ID NO:15 or SEQ ID NO:17, or comprises or consists of an amino acid sequence having at least 75%, 80%, 85%, 90%, 95%, 96%, 97%, 98% or at least 99% identity with SEQ ID NO:15 or SEQ ID NO:17.

In another particular embodiment, the chimeric construct comprises from N-terminus to C-terminus:
the targeting moiety;
optionally a linker; and
the IL-2 moiety.

In a preferred embodiment, the chimeric construct comprises from N-terminus to C-terminus:
the targeting moiety.
optionally a linker;
the moiety having dimerization properties, such as a of C4BPβ or a functional fragment thereof;
optionally a linker; and
the IL-2 moiety

### Homodimer and Heterodimer Constructs

It is herein described a method for producing a recombinant dimer protein comprising:
a) transfecting host cells with a vector allowing expression of a nucleotide sequence coding for a chimeric construct that is a fusion polypeptide comprising i) at least one interleukin 2 (IL2) moiety, ii) a moiety that is capable of forming a dimeric protein, such as a beta chain of the C4b-binding protein (C4BPβ) or at least one fragment or functional variant thereof, and iii) at least one targeting moiety as described above;
b) culturing transfected cells under conditions which are suitable for expressing the nucleotide sequence coding for the fusion polypeptide and the covalent association of two fusion polypeptides in vivo to form a dimeric protein;
c) recovering, and preferably purifying, the dimeric proteins formed.

The transfected cells preferably do not contain any nucleic acid allowing expression of a nucleotide sequence coding for the C-terminal fragment of the alpha chain of the C4BP protein involved in polymerization of the C4BP protein.

In a particular embodiment, it is herein described a method for producing heterodimers, said method comprising:
a. transfecting host cells with one or more vectors to allow the expression of one or more nucleotide sequences coding for:
i. a first fusion polypeptide comprising i) at least one interleukin 2 (IL2) moiety, and ii) a moiety that is capable of forming a dimeric protein, such as a beta chain of the C4b-binding protein (C4BPβ) or at least one fragment or functional variant thereof; and
ii. a second fusion polypeptide, comprising i) at least one targeting moiety as described above, such as a E06 scFv or a functional variant thereof, and ii) a moiety that is capable of forming a dimeric protein, such as a beta chain of the C4b-binding protein (C4BPβ) or at least one fragment or functional variant thereof;
b. culturing transfected cells under conditions appropriate for expressing the nucleotide sequence or sequences coding for the first and second fusion polypeptides and association of two fusion polypeptides in vivo to form a heterodimeric protein;
c. recovering, and preferably purifying, the heterodimeric proteins formed.

In a particular embodiment, it is herein described a method for producing heterodimers, said method comprising:
a. transfecting host cells with one or more vectors to allow the expression of one or more nucleotide sequences coding for:
i. a first fusion polypeptide comprising i) at least one interleukin 2 (IL2) moiety, ii) a moiety that is capable of forming a dimeric protein, such as a beta chain of the C4b-binding protein (C4BPβ) or at least one fragment or functional variant thereof, and iii) at least one targeting moiety as described above such as a E06 scFv or a functional variant thereof; and
ii. a second fusion polypeptide, comprising i) at least one heterologous polypeptide wherein the heterologous polypeptide is defined as being different from the interleukin 2 (moiety) of the first fusion polypeptide, ii) a moiety that is capable of forming a dimeric protein, such as a beta chain of the C4b-binding protein (C4BPβ) or at least one fragment or functional variant thereof, and iii) optionally at least one targeting moiety as described above such as a E06 scFv or a functional variant thereof;
b. culturing transfected cells under conditions appropriate for expressing the nucleotide sequence or sequences coding for the first and second fusion polypeptides and association of two fusion polypeptides in vivo to form a heterodimeric protein;
c. recovering, and preferably purifying, the heterodimeric proteins formed.

Preferably, in the second fusion polypeptide, C4BPβ or said fragment is fused to the C-terminal end of the heterologous polypeptide.

The term “different” when referring to the heterologous polypeptide means a polypeptide which has a primary amino acid sequence that is different by at least one amino acid from the primary sequence of the interleukin 2 (moiety) of the first fusion polypeptide. Alternatively, the term “different” also covers heterologous polypeptides having the same primary sequence but having different post-translational modifications, for example in terms of acetylation, amidation, biotinylation, carboxylation, hydroxylation, methylation, phosphorylation or sulfatation, or by adding lipids (isoprenylation, palmitoylation and myristoylation), glucides (glycosylation) or polypeptides (ubiquitination).

In a preferred embodiment, the heterologous polypeptide is not IL2.

Such heterodimer proteins are also part of the invention.

In a particular embodiment, the host cell allows co-expression of the two fusion polypeptides, a first fusion polypeptide A comprising i) at least one interleukin 2 (IL2) moiety, ii) a beta chain of the C4b-binding protein (C4BPβ) or at least one fragment or functional variant thereof that is capable of forming a dimeric protein and iii) at least one targeting moiety as described above such as a E06 scFv or a functional variant thereof; and a second fusion polypeptide A, comprising i) at least one interleukin 2 (IL2) moiety, ii) a beta chain of the C4b-binding protein (C4BPβ) or at least one fragment or functional variant thereof that is capable of forming a dimeric protein, and iii) at least one targeting moiety as described above such as a E06 scFv or a functional variant thereof. In this particular embodiment, co-expression of the two fusion polypeptides can also allow the production of homodimers A-A.

In a particular embodiment, the host cell allows co-expression of the two fusion polypeptides, a first fusion polypeptide A comprising i) at least one interleukin 2 (IL2) moiety, ii) a beta chain of the C4b-binding protein (C4BPβ) or at least one fragment or functional variant thereof that is capable of forming a dimeric protein and iii) at least one targeting moiety as described above such as a E06 scFv or a functional variant thereof; and a second fusion polypeptide B, comprising i) at least one heterologous polypeptide, ii) a beta chain of the C4b-binding protein (C4BPβ) or at least one fragment or functional variant thereof that is capable of forming a dimeric protein, and iii) at least one targeting moiety as described above such as a E06 scFv or a functional variant thereof; wherein the heterologous polypeptide is defined as being different from the interleukin 2 (moiety) of the first fusion polypeptide. In this particular embodiment, co-expression of the two fusion polypeptides can also allow the production of homodimers A-A and B-B and the production of heterodimers A-B.

It is also provided a recombinant eukaryotic cell allowing synthesis of a dimer or heterodimer protein as defined above, and obtainable by carrying out step a) of the production method defined above. Greater details for the production in host cells are described below.

### Production Methods

The chimeric construct, which is in the form of a fusion protein, and the homo- or heterodimers can be produced by DNA recombinant technique in a suitable expression vector or by a RNA molecule.

The expression vector is selected as a function of the host cell into which the construct is introduced. Preferably, the expression vector is selected from vectors that allow expression in eukaryotic cells, especially from chromosomal vectors or episomal vectors or virus derivatives, in particular vectors derived from plasmids, yeast chromosomes, or from viruses such as baculovirus, papovirus or SV40, retroviruses, Adenoviruses, Adeno-associated Viruses, or combinations thereof, in particular phagemids and cosmids. In a particular embodiment, it is a vector allowing the expression of baculovirus, capable of infecting insect cells.

If necessary, the sequence coding for the fusion polypeptide also comprises, preferably in its 5′ portion, a sequence coding for a signal peptide for the secretion of fusion polypeptide. Conventionally, the sequence of a signal peptide is a sequence of 15 to 20 amino acids, rich in hydrophobic amino acids (Phe, Leu, Ile, Met and Val).

The vector comprises all of the sequences necessary for the expression of the sequence coding for the fusion polypeptide. In particular, it comprises a suitable promoter, selected as a function of the host cell into which the construct is to be introduced.

Within the context of the invention, the term “host cell” means a cell capable of expressing a gene carried by a nucleic acid which is heterologous to the cell and which has been introduced into the genome of that cell by a transfection method.

Preferably, a host cell is a eukaryotic cell. A eukaryotic host cell is in particular selected from yeast cells such as S cerevisiae, filamentous fungus cells such as Aspergillus sp, insect cells such as the S2 cells of Drosophila or sf9 of Spodoptera, mammalian cells and plant cells. Mammalian cells which may in particular be cited are mammalian cell lines such as CHO, COS, HeLa, C127, 3T3, HepG2 or L (TK-) cells. In a preferred implementation, said host cells are selected from eukaryotic cell lines, preferably Sf9 insect cells. Methods for preparing recombinant dimeric proteins in sf9 insect cells are described in U.S. Pat. No. 7,884,190. Any transfection method known to the skilled person for the production of cells expressing a heterologous nucleic acid may be used to carry out step a) of the method. Transfection methods are, for example, described in Sambrook et al, 2001, “Molecular Cloning: A Laboratory Manual”, 3rd edition, Cold Spring Harbor Laboratory Press, Cold Spring Harbor, N.Y.

Alternatively, the chimeric construct can be produced by chemical peptide synthesis. For instance, the protein can be produced by the parallel synthesis of shorter peptides that are subsequently assembled to yield the complete sequence of the protein with the correct disulfide bridge. A synthesis of IL-2 is illustrated for instance in Asahina et al., Angewandte Chemie International Edition, 2015, Vol. 54, Issue 28, 8226-8230, the disclosure of which being incorporated by reference herein.

In another embodiment, the chimeric protein may be expressed in vivo, after administering the subject with a nucleic acid encoding said chimeric protein. In a preferred embodiment, the nucleic acid is carried by an RNA or a viral vector, such as an adeno-virus associated virus (AAV).

### Formulations and Routes of Administration

It is also provided a pharmaceutical composition comprising a chimeric construct, a nucleic acid, a vector or a protein as described herein, preferably in association (e.g., in solution, suspension, or admixture) with a pharmaceutically acceptable vehicle, carrier or excipient.

Suitable excipients include any isotonic solution, saline solution, buffered solution, slow release formulation, etc. Liquid, lyophilized, or spray-dried compositions are known in the art and may be prepared as aqueous or nonaqueous solutions or suspensions. Preferably the pharmaceutical compositions comprise appropriate stabilizing agents, buffering agents, bulking agents, or combinations.

The pharmaceutical composition may further contain another active ingredient, or may be administered in combination with any other active ingredient.

The pharmaceutical composition may be administered using any convenient route, including parenteral, e.g. intradermal, subcutaneous, or intranasal route. The subcutaneous route is preferred. Oral, sublingual or buccal administrations are also encompassed.

An example of a formulation suitable for a subcutaneous injection is described in international patent application WO2017/068031.

Treatment of Auto-Immune and/or Inflammatory Disorders

The pharmaceutical compositions described herein are useful in methods for treating an auto-immune and/or inflammatory disorder, such as systemic lupus erythematous, type I diabetes, HCV-related vasculitis, uveitis, myositis, systemic vasculitis, psoriasis, allergy, asthma, Crohn's disease, multiple sclerosis, rheumatoid arthritis, atherosclerosis, autoimmune thyroid disease, auto-inflammatory diseases, neuro-degenerative diseases, including Alzheimer's disease and amyotrophic lateral sclerosis, acute and chronic graft-versus-host disease, spontaneous abortion and allograft rejection; solid organ transplantation rejection, vasculitis, inflammatory bowel disease (IBD), and allergic asthma; spondyloarthritis or ankylosing Spondylitis; Sjogren's syndrome, Systemic sclerosis, Alopecia aerate, or Ulcerative Colitis.

It is also suitable in conditions where the activation of Tregs tissue regeneration properties are desired, such as in muscle diseases, neurodegeneration, cardiac or other tissues infarction.

In a preferred embodiment, it is herein described a method of treatment of an auto-immune and/or inflammatory disorder, comprising administering the composition once or twice a week, or even once or twice a month, preferably by subcutaneous route. In one embodiment, a dosage of less than 30 MIU/day, preferably less than 20 MIU/day is preferred, advantageously less than 10 MIU/day, or between 1 MIU/day and 8 MIU/day. In another particular embodiment, a dose of between 1 and 5 MIU/day, preferably from 0.1 to 3.5 MIU/day is used

Generally speaking, doses that allow a 1.2, 1.5, 2, 3, 4 or 5-fold increase of the number of Tregs are preferred. The standard measure of an amount IL-2 is the International Unit (IU), which technically is not a fixed weight but the amount that produces a fixed biological effect in a specific cell proliferation assay, as determined by the World Health Organization (WHO). The reason is that i) the weight varies depending on the exact sequence of the molecule and its glycosylation profile, and ii) what matters is the activity, not the weight of the molecule.

The principle of the International Unit is precisely to provide a standard to which any IL-2 molecule can be compared (regardless of their source, or their sequence, including wild-type or active variant sequences).

In practice, the WHO provide ampoules containing an IL-2 molecule that has been calibrated and serves as the reference to determine the dosage of a given preparation of IL-2 (again regardless of the source or sequence of said IL-2) defined by its potency. For instance, to determine the dosage of a given preparation of IL-2, the biological activity of the candidate IL-2 preparation is measured in a standard cell proliferation assay using an IL-2 dependent cell line, such as CTLL-2, and compared with the biological activity of the standard. The cells are grown in the presence of different doses of the standard. A dose-response effect of IL-2 is established, where the dose of IL-2 is plotted on the X axis as IU and the measure of proliferation (pr) is on the Y axis. When one wants to determine the activity of any IL-2 product of unknown activity, the product is used to grow the IL-2 dependent cells and the proliferation is measured. The pr value is then plotted on the Y axis and from that value a line parallel to the X axis is drawn. From the point of intersection of this line with the dose response line, a line parallel to the Y axis is then drawn. Its intersection with the X axis provides the activity of the candidate IL-2 product in IU.

Any change of the WHO standard ampoules does not impact the International Unit nor the determination of a dosage of any IL-2 preparation.

The 1st standard (WHO international Standard coded 86/504, dated 1987) contained a purified glycosylated IL-2 derived from Jurkat cells and was arbitrarily assigned a potency of 100 IU/ampoule. As the stocks of the 1st international standard (IS) were running low, the WHO had to replace it. The WHO provided another calibrated IL-2 ampoule, this time produced using E. coli. The 2nd standard ampoules contained 210 IU of biological activity per ampoule. The change of standard ampoules does not mean that the IU changes. So, determining the dosage of a test IL-2 preparation will not vary whether one uses the 1st standard ampoule or the 2nd standard ampoule, or a subsequent standard ampoule, as a reference.

In one embodiment, a chronic administration is implemented, e.g. comprising administration once every 3 days to once every three months. Such sequences of administration may be repeated if needed.

In another embodiment, the IL-2 is given every other day for 1 to 2 weeks, in cycles that can be repeated after break of administration that can last from 3 days to 3 months, preferably from one to 4 weeks.

In another embodiment, the treatment may comprise a first course that is also designated as an induction course, and a second course, that is maintenance course.

In a particular embodiment, the treatment may comprise at least a first course wherein the pharmaceutical composition is administered once per day during at least about 2 or 3 consecutive days, preferably during 3 to 7, still preferably during 4 to 5 consecutive days, preferably followed by a maintenance dose, e.g. after about six days or about 1 to about 4 weeks.

The maintenance dose may be typically administered during at least one month, preferably at least about 3 months, still preferably at least about 6 months. In a preferred embodiment, the maintenance dose is administered between about 3 months and about 12 months, preferably between about 6 months and about 12 months.

In a preferred embodiment, the maintenance treatment consists of an administration of the pharmaceutical composition once or twice a week, or every one or two weeks, or once a month. In a preferred embodiment, the maintenance treatment consists of an administration of interleukin-2 once or twice a week, every one or two weeks, or once a month during a period of at least one month, preferably from about 3 months to about 12 months.

Preferably the maintenance dosage is substantially the same as the first course dosage, or it can be a lower or higher dosage.

### Treatment of Cancers

The pharmaceutical compositions described herein are useful in methods for treating a cancer. In some embodiments, the subject is suffering from locally advanced or metastatic cancer. In some embodiments, the cancer is a solid tumor. In some embodiments, the cancer is colon cancer, lung cancer, ovarian cancer, gastric cancer, bladder cancer, pancreatic cancer, endometrial cancer, breast cancer, kidney cancer, esophageal cancer, or prostate cancer. In one embodiment, a dosage of less than 30 MIU/day, preferably less than 20 MIU/day is preferred, advantageously less than 10 MIU/day, or between 3 MIU/day and 5 MIU/day. In other embodiments, 400,000-750,000 IU/kg or 550,000-750,000 IU/kg, preferably 600,000-700,000 IU/kg, IL2 is administered. The dosage may be similar to, but is expected to be less than, that prescribed for PROLEUKIN®.

The compositions can be administered once from one or more times per day to once or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject can include a single treatment or, can include a series of treatments.

The examples of protocols described above in connection with auto-immune and/or inflammatory disorders may be applied identically or similarly for use in treating a cancer. Alternatively, in another example, the compositions may be administered every 8 hours for five days, followed by a rest period of 2 to 14 days, e.g., 9 days, followed by an additional five days of administration every 8 hours. In some embodiments, administration is 3 doses administered every 4 days.

### EXAMPLES

### Example 1: In Vitro and In Vivo Targeting

### Materials and Method

### Fusion Proteins

Lentiviral vectors were used for production of IL-2 fusion proteins and targeted proteins. Briefly, human IL-2-C4bpß fused to scFvE06 capable of binding to phosphocholine oxidized lipid (IL-2-C4bpß-E06 or IL-2N88R-C4bpß-E06) were integrated in a lentiviral plasmid under the spleen focus-forming virus (SFFV) promoter. “IL-2-C4bpß-E06” (SEQ ID NO:19) is human IL-2 fused to the N-terminus of C4BPß, C4BPß being fused to the N-terminus of scFVE06. “IL-2N88R-C4bpß-E06” (SEQ ID NO:20) is a mutated IL-2 fused to the N-terminus of C4BPß, C4BPß being fused to the N-terminal region of scFVE06.

HEK 293T cells were transfected at 70% confluence with lentiviral plasmids using polyethyleneimine (PEI) and cultured for 24-30H in a serum-free medium. Supernatants were then filtered and concentrated by ultracentrifugation and resuspended in appropriate buffer before conservation at −80° C. To obtain stable transfected cells, HEK 293T cells were infected with lentivirus at different multiplicity of infection (MOI) before cell sorting of GFP+ cells to ensure 100% of cells producing IL-2 fusion proteins. Stable cell lines were cultured for 48H in a serum-free medium and supernatants were harvested, and purified using HisPur Ni-NTA Resin (Thermofisher) enables effective immobilized metal affinity chromatography (IMAC) purification before concentration, buffer exchange and validation by SDS-PAGE.

pSTAT5 Analyses on Human

Human blood samples from healthy volunteers were obtained from Etablissement Français du Sang (EFS) at Saint Antoine Hospital in Paris, France. Informed consent was obtained from each volunteer. The effects of human IL-2, fusion proteins and targeted proteins on the induction of STAT5 phosphorylation (pSTAT5) were assessed in human CD4+ regulatory T cells (Treg; CD4+Foxp3+CD127lo/−), CD4+ conventional T cells (Tconv; CD4+Foxp3−), CD8+ T cells and natural killer cells (CD3−CD56+) using flow cytometry. Ten-fold dilution of human IL-2, fusion and targeted proteins mixed with 100 μl of whole blood for 15 min at 37° C. were performed before pSTAT5 staining using the Phospho-Epitopes exposure kit (PERFIX EXPOSE kit, Beckman Coulter). In conjunction with the intracellular pSTAT5 signal, surface markers allowed the comparison of activity and the calculation of half maximal effective concentration (EC₅₀) values for different lymphocyte cell populations.

### ELISA

For targeted proteins detection, plasmatic and protein levels were measured following the same protocol as described here above, except for capture anti-IL-2 monoclonal antibody (MQ1-17h12) or Phosphocholine-BSA (LGC Biosearch technologies) coated at 1 μg/mL and detection performed using anti-His antibody (1:1000; Thermofisher) at 1 μg/mL before revelation with ultrasensitive streptavidin-HRP (1:2000; Sigma Aldrich).

### Flow Cytometry and Antibodies

### Whole Blood:

After mice blood hemolysis, isolated immune cells were stained with the following antibodies at predetermined optimal dilutions for 20 minutes at 4° C.: CD3-PEfluor610 (Invitrogen), CD4V500 (BD Bioscience), CD8AF700 (BD Pharmingen), CD25PeCy7 (Invitrogen), NKp46eF660 (eBioscience), and CD19eF780 (eBioscience). Intracellular detection of Foxp3-FITC (eBioscience) was performed on fixed and permeabilized cells using FoxP3 staining buffer kit (eBioscience FoxP3/Transcription). Cells were acquired on cytoflex S (Beckman Coulter) and analyzed with FlowJo software. Dead cells were excluded by forward/side scatter gating. CD4+ Tregs were defined as CD4+CD25+Foxp3+ cells, CD4+ Teffs as CD4+CD25+Foxp3− cells also called Tconv CD4+CD25+, CD8+ Tregs were defined as CD8+CD25+Foxp3+ cells, CD8+ Teffs also called Tconv CD8+CD25+, as CD8+CD25+Foxp3+ cells, Natural killer cells as Nkp46+ cells and B cells as CD19+ cells. For STAT5 assay, human whole blood was incubated with the following antibodies against membrane proteins for 15 min: CD3-FITC, CD127-PC7, CD4-PB and CD8-KO, CD45RA-AF700 and CD56-APC-eFluor780. After fixation and permeabilization, cells were stained with intracellular p-STAT5-PE and Foxp3-APC antibodies.

### Colitis Inflammatory Experimental Model

C57BL/6 mice were immunized by oral administration of 2.5% of Dextran Sulfate Sodium (DSS; Sigma) in drinking water during 6 days. Mice were monitored daily for body weight, consistency and presence of blood in their stool during 10 days. The following scoring system has been used to evaluate the severity:
Loss of body weight between 0-5%: 1/5-10%: 2/10-15%: 3/and over 15%: 4.
Stool consistency: normal stool: 0/formed but soft stool: 1/loose stool: 2/mild diarrhea: 3/watery diarrhea: 4.
1 Gross bleeding: absence: 0/presence: 2/gross bleeding: 4.

### Results

Ability of Targeted Fusion Proteins to Recognize their Target, Dimerize and Sustain their Biological Activity In Vitro

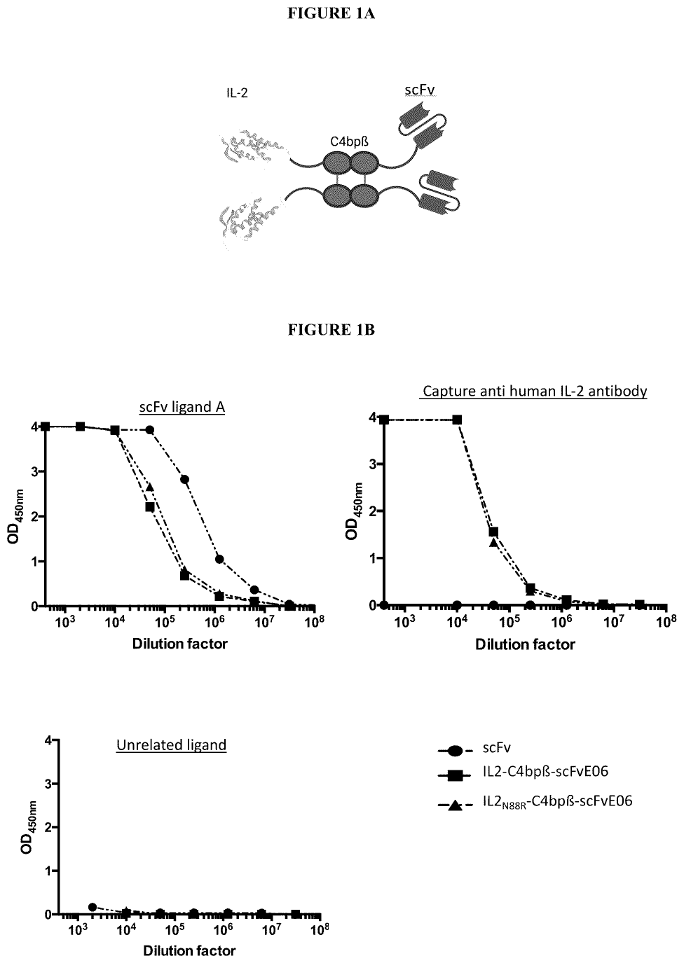

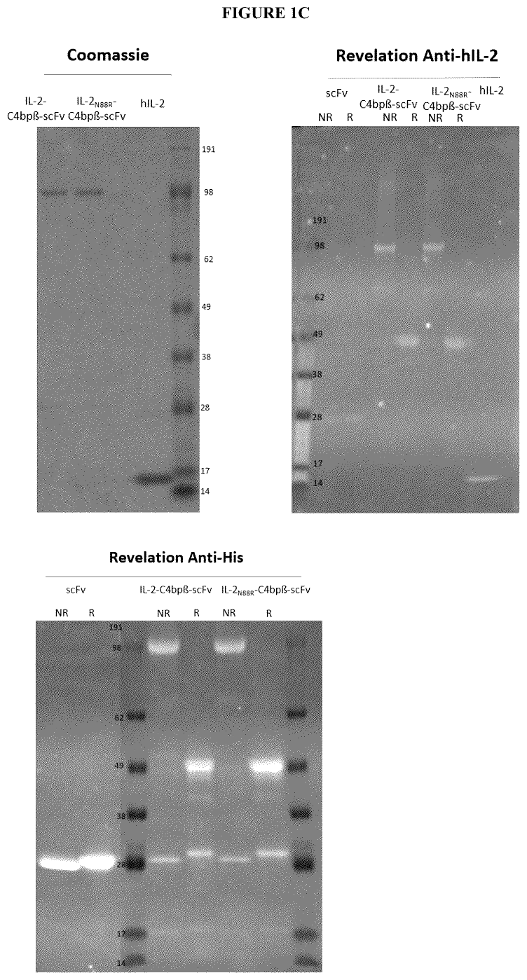

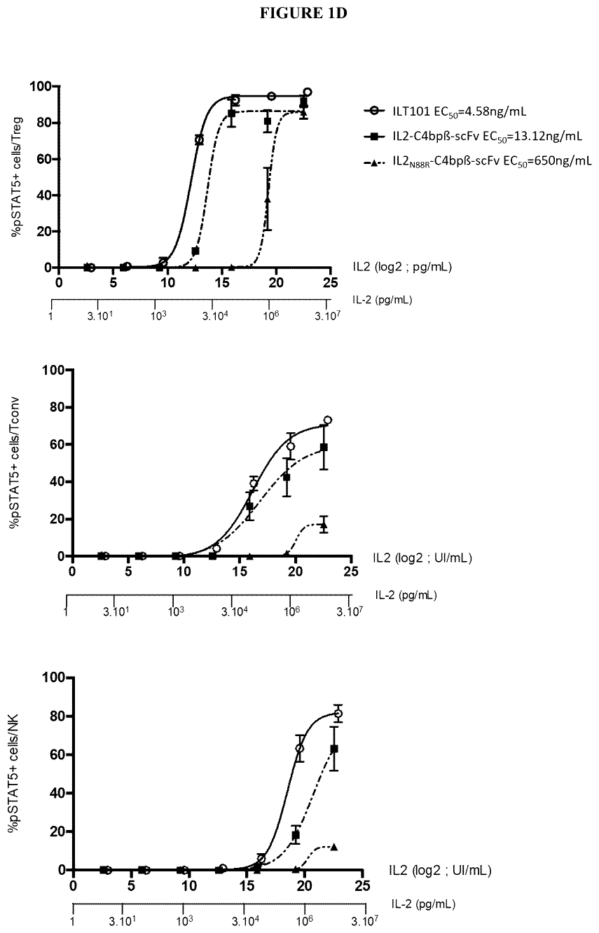

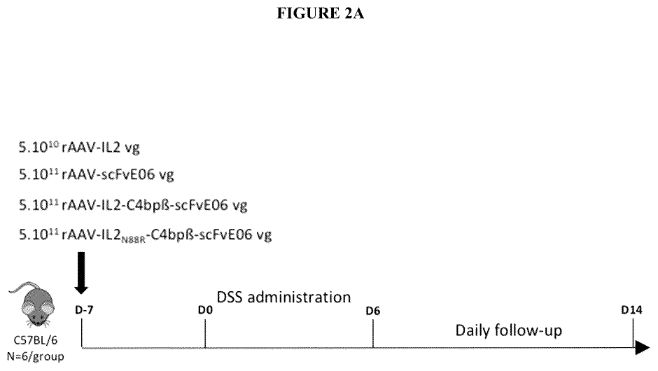

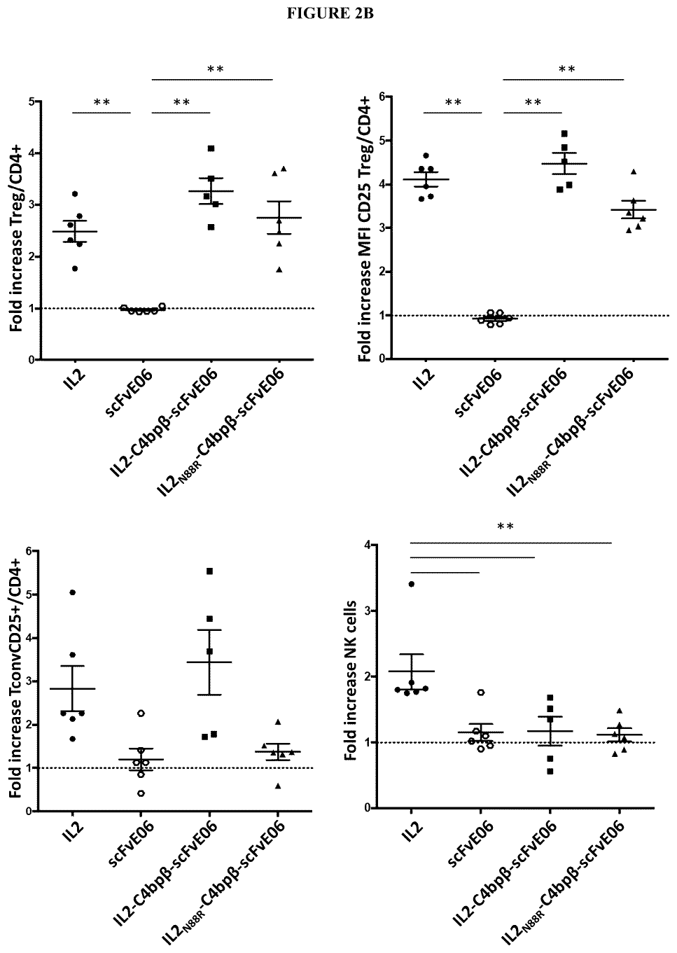

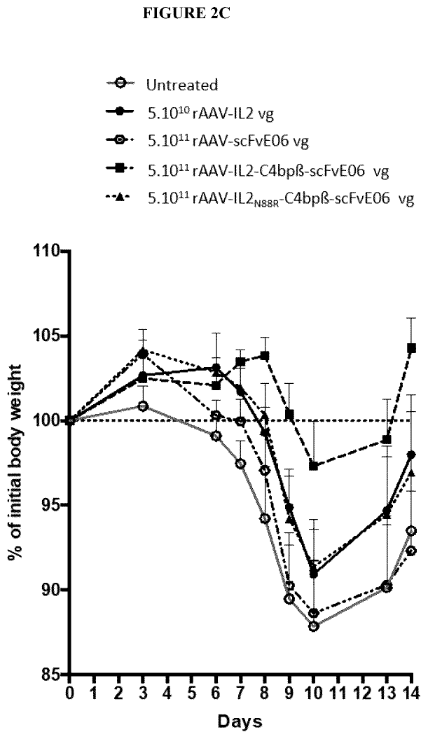

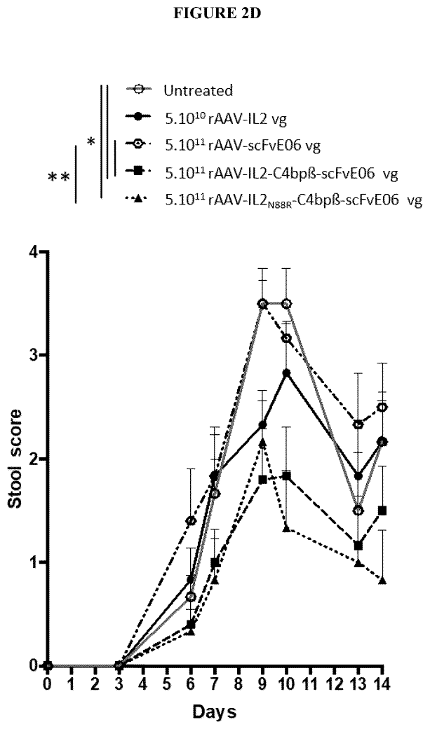

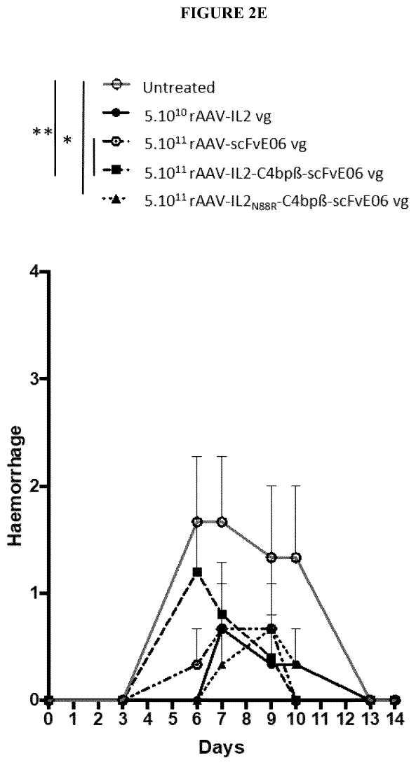

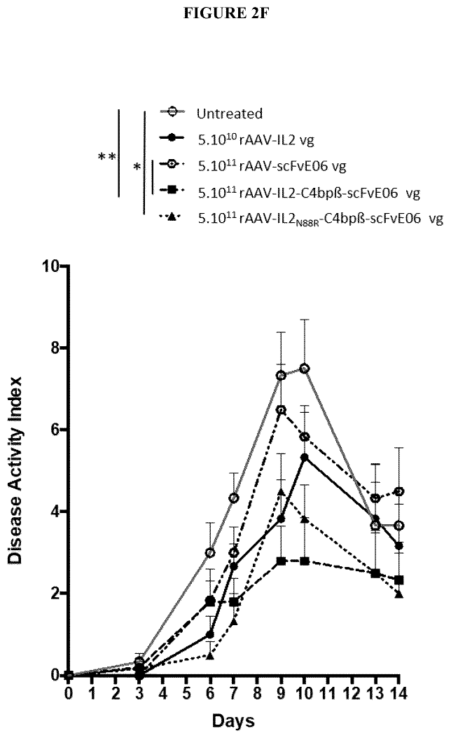

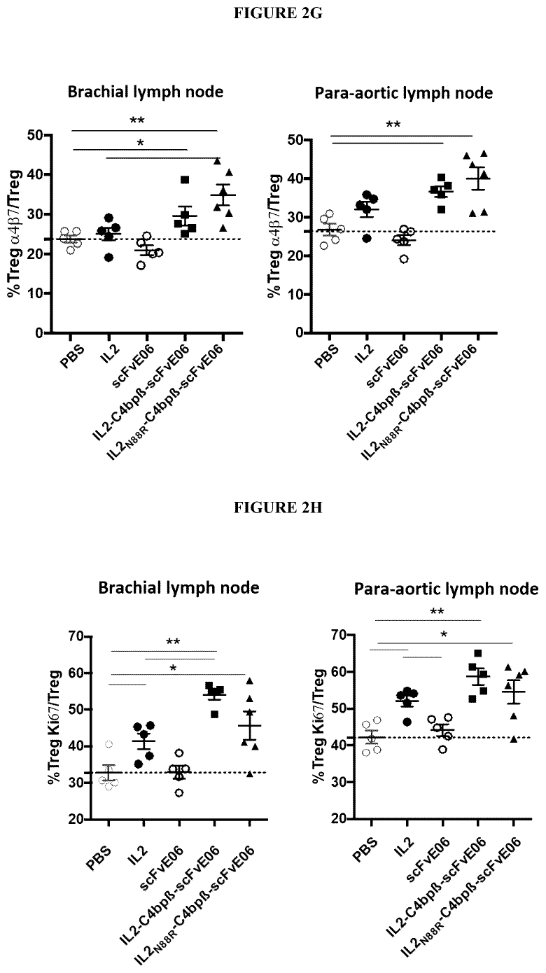

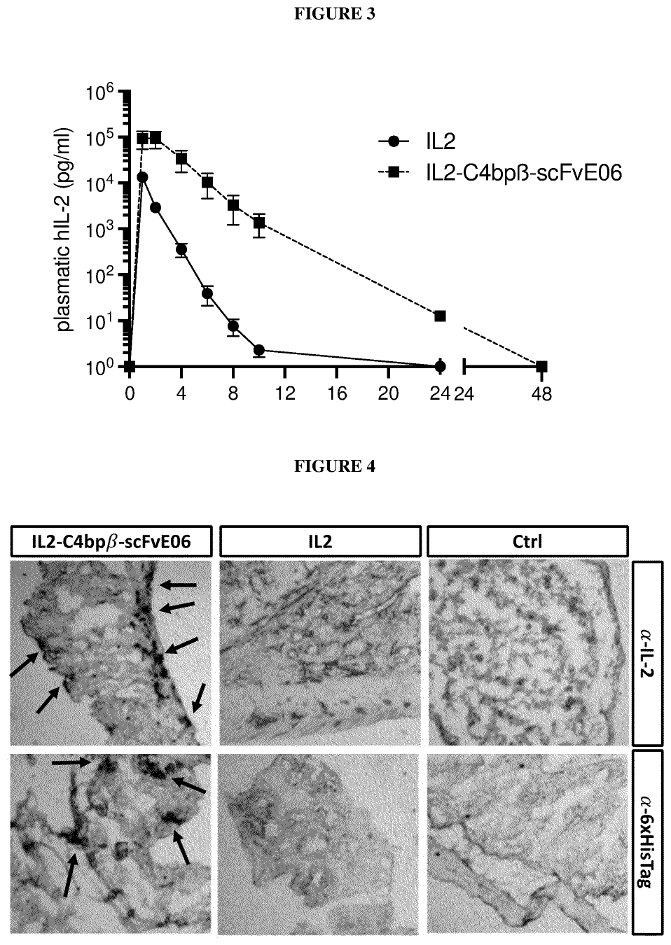

IL-2 construct was combined with tissue-selective moiety to target inflammatory tissues without altering its functions. In details, IL-2-C4bpß or IL-2N88R-C4bpß were fused at the C-terminal part of C4bpß to the scFvE06, which is known to be specific for oxidized phospholipids such as phosphocholine (PC) (FIG. 1.A).

To determine in vitro binding of the targeted proteins, PC-BSA, anti-hIL-2 antibody or BSA were coated before addition of either targeted proteins or scFvE06 supernatants and revelation using anti-His-tag antibody (FIG. 1.B). After PC-BSA coating, the three constructs were detected meaning that proteins are capable of binding to PC-BSA. In addition, after hIL-2 coating, only targeted fusion proteins were detected which highlighted the good conformation of targeted proteins being recognized by anti-IL-2 and anti-His antibodies. Finally, no detection of targeted proteins was observed after BSA-coating confirming that there is no non-specific binding and that scFvE06 is specific of PC.

After filtration, purification and concentration, recombinant targeted proteins were also characterized after SDS-PAGE followed by Coomassie blue staining and Western Blot (FIG. 1.C). A unique signal is detected after Coomassie staining for each protein around 98 kDa meaning that there is only one major protein in the samples under non-reduced condition (FIG. 1.C). Whether they are revealed by a primary anti-human IL-2 or a primary anti-histidine antibody, under heat and reductive conditions, bands are observed for IL-2-C4bpß-E06 and IL-2_{N88R}-C4bpß-E06 characteristic of the protein in its monomeric (50 kDa) and dimeric (100 kDa) forms. In addition, separately from the conditions, a single band is also observed at 25 kDa for each protein after primary anti-histidine antibody revelation characteristic of the scFvE06. To compare, native human IL-2 is revealed only using primary anti-human IL-2 antibody with a band observed at 17 kDa.

Once again, human whole blood assay measuring STAT5 phosphorylation was performed to determine the effect of the targeted fusion proteins on the different cell types expressing IL-2 receptor. Regulatory T cells EC₅₀ were about 5 ng/ml for native hIL-2, 13 ng/ml for IL-2-C4bpß-E06 and 650 ng/mL for IL-2_{N88R}-C4bpß-E06 meaning that 3 times more IL-2-C4bpß-E06 and 130 times more IL-2_{N88R}-C4bpß-E06 were needed to obtain equivalent pSTAT5 responses on Treg cells (FIG. 1.D). Differences in pSTAT5 profiles were also observed on Tconv, CD8+ cells and NK cells populations.

Indeed, a reduced pSTAT5 response is observed with the targeted fusion proteins at doses up to at least 1000 ng/mL, whereas a response is obtained with native hIL-2 on these effector compartments from a dose of 100 ng/mL. For example, less than 20% of Tconv, CD8+ and NK cells are activated in response to approximately 10000 ng/ml of IL-2N88R-C4bpß-E06. These results highlight Tregs selectivity of the targeted fusion protein due to the reduction binding affinity to the dimeric receptor.

### Proof of Efficacy of Targeted Fusion Proteins in a Colitis Inflammatory Experimental Model

The targeted fusion proteins were evaluated in a colitis inflammatory experimental model for their ability to control the severity of the disease (FIG. 2.A). To obtain similar expansion of Tregs, rAAV doses were adapted from 5·10¹⁰ rAAV vg in IL-2 treated group to 5·10¹¹ rAAV vg for scFvE06, IL-2-C4bpß-scFvE06 and IL-2_{N88R}-C4bpß-scFvE06 treated ones.

Seven days after injections, Tregs cells were expanded by 2.5 to 3-fold times in groups treated with rhIL-2 or targeted fusion proteins and activated considering the 3 to 4 times increase of CD25 MFI (FIG. 2.B). The mutated protein didn't expand effector populations whereas hIL-2 and IL-2-C4bpß-scFvE06 treatments result in effector populations expansion by 3-fold times. In addition, NK cells were only expanded in mice treated with hIL-2 even if the dose was ten times lower.

As expected, no significant changes were observed on these four parameters after 5·10¹¹ rAAV vg scFvE06 injection.

After six days of DSS administration, untreated mice developed important clinical manifestations with significant loss around 10% of their initial body weight associated with mild diarrhea (stool score of 3.5) and the presence of blood in the stools explained by the severe colon inflammation (FIG. 2 C, D, E).

The disease activity index (DAI) which corresponds to the addition of these symptoms increased from day 3 to 10 and reached a score of 8 out of 12 before decreasing until day 14 (FIG. 2.F). To compare, mice treated with scFvE06 had similar loss body weight and stool consistency but almost no haemorrhage which explained the slight decrease of the DAI overtime compared to untreated mice. A treatment by hIL-2 partially control the clinical manifestations with less body weight, very soft but not watery and almost no haemorrhage explaining a maximum DAI score of 5 ten days after first DSS administration. Interestingly, mice treated with targeted fusion proteins had significantly less intestinal clinical manifestations with formed and partially soft stools (maximum score of 2) and almost no haemorrhage. Finally, DAI never exceeded a score of 4 in each targeted fusion groups confirming the therapeutic benefit of these two proteins in the treatment of colitis.

Interestingly, in brachial and para-aortic lymph nodes, Tregs expressing integrin α4ß7, required to pass through intestinal barrier, are present in better proportions after treatment with targeted fusion proteins than native IL-2. These observations suggest that the numbers of Treg recruited to the inflammatory site are increased in targeted proteins groups and indirectly that the amount of IL-2 in the intestine of mice treated with these targeted proteins is higher, highlighting the targeting capabilities of IL-2-C4bpß-scFvE06 and IL-2_{N88R}-C4bpß-scFvE06. Similarly, there are more Tregs expressing Ki67 in these two groups compared to native IL-2 meaning that these Tregs proliferate more in these two groups.

### Example 2: In Vivo Pharmacokinetics

### Methods

### PK Analysis

7 weeks old female C57Bl/6 mice were injected subcutaneously with 50 KIU of human IL-2 or IL-2-C4bpß-scFvE06 in 100 μl. 100 μl of blood were up-taken at different timepoint for hIL-2 dosage by ELISA: Baseline, 1h, 2h, 4h, 6h, 8h, 10h, 24 h and 48h. Plasma samples were 10-fold serially diluted in appropriate buffer until 1/1000. Elisa was performed using supplier recommendations and plates were read at 450 nm. Concentration of hIL-2 in both groups were calculated using an appropriate standard curve.

### In Vivo Binding of IL-2-C4bpß-scFvE06

### Psoriasis Model

Psoriasis model in mice was induced by daily application of a 5% Imiquimod cream on mice ears, for 6 days. As internal control, the control-lateral ear of each mouse was applied with Vaseline following the same schedule. 10 days before Psoriasis induction, 8 weeks old female Balb/c mice were injected IP with 5·10¹¹ vg AAV coding for IL-2-C4bpß-scFvE06. Control mice of psoriasis were not treated. At the end of the experiment, 6 days after the first application, mice were euthanized, ears were removed and immediately frozen in OCT. 8 μm cross-sections of the ears were then made using cryostat before fixation, permeabilization and staining with DAPI (nuclear staining) and anti-6×-HisTag-Cy5 for IL-2-C4bpß-scFvE06 detection. Sections were analyzed using a fluorescence microscope.

### DSS-Induced Colitis Model

DSS-induced colitis model in mice was induced by addition of 3% Dextran-Sulfate Sodium in drinking water for 6 days. 10 days before Colitis induction, 8 weeks old female C57Bl/6 mice were injected IP with 5.1011 vg AAV either coding for IL2 or IL-2-C4bpß-scFvE06. A group of control mice were added and was not treated. At the end of the experiment, 10 days after induction, at the maximum of inflammation, mice were euthanized, colons were removed and immediately frozen in OCT. 8 μm cross-sections of the ears were then made using cryostat before fixation, permeabilization and staining with anti-IL2-HRP or anti-6×-HisTag-HRP for IL-2-C4bpß-scFvE06 detection. Histochemistry brown/black coloration was obtain after incubation with Metal enhanced DAB substrate.

### Results

### Pharmacokinetics

In order to determine if we increased half-life of the molecule, a pharmacokinetic experiment was performed in mice after a single injection of IL2 or IL-2-C4bpß-scFvE06, 50 kIU injected SC (FIG. 3). IL2 increased rapidly with Tmax reached after 1h, before a very quick decrease until 10h where IL2 were almost no detectable. On the other hand, IL-2-C4bpß-scFvE06 increased more slowly, reaching Tmax between 1h and 4h post injection. The decrease of plasmatic concentration was also slower, with molecule still detectable at 24h. This result demonstrates that IL-2-C4bpß-scFvE06 has an increased half-life, calculated about 3-fold increase in this setting. This could allow an increase in the effective duration of IL-2 on Tregs, with the increase in Tregs being maintained over a longer term.

### Proof of Concept of In Vivo Targeting of IL-2-C4bpß-scFvE06

We developed 2 models of inflammatory diseases, Psoriasis and Colitis.

Psoriasis was induced by local application of a 5% imiquimod cream in one ear, when the control-lateral ear was used as internal control of inflammation and applied with Vaseline. In these conditions, one ear develops inflammation which should be targeted by IL-2-C4bpß-scFvE06 whereas the other one does not develop inflammation and should not be particularly targeted. Briefly, 6 days after daily application of imiquimod and Vaseline, mice were euthanized and ears were removed, frozen, cross-sections was performed and stained with DAPI and anti-6×-HisTag coupled with Cyanine 5 Fluorochrome for fluorescence microscopy. While control mice, not treated, did not shown any 6×-HisTag staining nor in the Vaseline-treated ear, nor in the imiquimod-treated ear, we observed a staining of our molecule in mice injected with AAV coding for IL-2-C4bpß-scFvE06, all around the ear where inflammation occurred. As control in the same mice, IL-2-C4bpß-scFvE06 was not detected in Vaseline-treated ears demonstrating the in vivo specific binding of inflamed tissue of our targeted molecule.

We further developed a DSS-induced colitis model to test our molecule in different conditions (FIG. 4). 3% DSS were added in drinking water for 6 days to induce colitis. At day 10, time where inflammation is maximum, mice were euthanized and colons were removed, frozen, cross-sections was performed and stained with anti-hIL2 or anti-6×-HisTag both coupled to HRP. Immunohistochemistry staining was performed using Metal enhance DAB substrate, where a black or dark brown coloration developed where the molecule was found. As a result, neither untreated control group, nor IL2-treated group demonstrated detection of IL2 or 6×-HisTag in colons. However, mice treated with IL-2-C4bpß-scFvE06 showed detection of both IL2 and 6×-HisTag in colon, demonstrating again the in vivo specific binding of IL-2-C4bpß-scFvE06 in inflammatory condition.

1. A chimeric construct comprising
i) at least one interleukin 2 (IL2) moiety; and
ii) at least one targeting moiety, which binds to an oxidized protein or oxidized lipid.

2. The chimeric construct of claim 1, wherein the targeting moiety binds to a pro-inflammatory oxidized protein or oxidized lipid.

3. The chimeric construct of claim 1, wherein the targeting moiety binds to an oxidation-specific epitope (OSE).

4. The chimeric construct of claim 1 wherein the targeting moiety binds (i) to a malondialdehyde (MDA) epitope, (ii) to a 2-(ω)-carboxyethyl) pyrrole (CEP) epitope, (iii) to a 4-hydroxynonenal (4-HNE) epitopes, or (iv) to an oxidized phospholipid (OxPL).

5. The chimeric construct of claim 1, wherein the targeting moiety is an antibody or an antibody fragment.

6. The chimeric construct of claim 5, wherein the targeting moiety is E06 scFv or a variant thereof that comprises:
a variable heavy chain (VH) domain comprising an amino acid sequence as shown in SEQ ID NO: 12, or an amino acid sequence having at least 75%, 80%, 85%, 90%, 95%, 96%, 97%, 98% or at least 99% identity with SEQ ID NO: 12; and
a variable light chain (VL) domain comprising an amino acid sequence as shown in SEQ ID NO: 11, or an amino acid sequence having at least 75%, 80%, 85%, 90%, 95%, 96%, 97%, 98% or at least 99% identity with SEQ ID NO:11.

7. The chimeric construct of claim 1, wherein said IL-2 moiety is human IL-2 or homologous variant thereof, wherein the variant has at least 85% amino acid identity with human wild-type IL-2, preferably wherein the variant is an active analogue of human IL-2 which has at least 90% amino acid identity with human wild-type IL-2.

8. The chimeric construct of any claim 1 wherein the IL2 moiety and the targeting moiety are fused in frame or through an amino acid linker.

9. The chimeric construct of claim 1, said chimeric construct further comprising a beta chain of the C4b-binding protein (C4BPβ) or at least one fragment or functional variant thereof that is capable of forming a dimeric protein.

10. The chimeric construct of claim 9, comprising a functional variant of C4BPβ which comprises:
a) a modified sequence of the fragment of C4BPβ, wherein less than 25 percent of the amino acids of the fragment, have been cut out or replaced, in which the cysteines located in positions 202 and 216 as well as at least 3 amino acids upstream and downstream of each cysteine have been conserved; or
b) a modified sequence of the fragment of the C4BPβ, wherein a cysteine responsible for dimerization is substituted with an amino acid, and another amino acid of the fragment is substituted with a cysteine; or
c) a sequence of the fragment of C4BPβ modified by insertion of a sequence which is heterologous to the beta chain, between the cysteines responsible for dimerization; or
d) a sequence of the fragment of C4BPβ modified by cutting out amino acids between the cysteines responsible for dimerization.

11. The chimeric construct of claim 9, wherein the IL-2 moiety is fused at the N-terminus of C4BPβ or said fragment thereof.

12. A nucleic acid encoding the chimeric construct of claim 1.

13. A vector comprising the nucleic acid of claim 12.

14. A host cell comprising the nucleic acid of claim 12.

15. A method for treating an auto-immune and/or inflammatory disease in a subject in need thereof, which method comprises administering the subject with the chimeric construct of claim 1.

16. The chimeric construct of claim 4, wherein the targeting moiety binds to a phosphocholine-containing oxidized phospholipid (PC-OxPL), an oxidized phosphatidylethanolamine (OxPE), an oxidized phosphatidylserine (OxPS) or an oxidized cardiolipin (OxCL).

17. The chimeric construct of claim 16, wherein the targeting moiety binds to a phosphocholine-containing oxidized phospholipid (PC-OxPL).

18. The chimeric construct of claim 1, wherein the targeting moiety is a single chain variable fragment (scFv).

19. The chimeric construct of claim 1, wherein the targeting moiety is selected from the group consisting of: a E06 antibody or a E06 antibody fragment; a LR04 antibody or a LR04 antibody fragment; a NA17 antibody or a NA17 antibody fragment; a E014 antibody or a E014 antibody fragment; a MDA2 antibody or a MDA2 antibody fragment; a IK17 antibody or a IK17 antibody fragment; a LR01 antibody or a LR01 antibody fragment, and functional variants thereof.

20. The chimeric construct of claim 1, wherein said IL-2 moiety is an IL2 mutein that comprises a substitution at position N88 of SEQ ID NO: 2.

21. The chimeric construct of claim 20, wherein said IL-2 moiety is an IL2 mutein that comprises a substitution N88R or N88D

22. The chimeric construct of claim 9, wherein the fragment of C4BPβ comprises, or consists of, amino acid residues 194 to 252 of C4BPβ or a longer fragment of C4BPβ that extends at the N-term up to at most amino acid 135.


SEQUENCE LISTING (20 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - protein, 252 aa, Homo sapiens
MFFWCACCLMVAWRVSASDAEHCPELPPVDNSIFVAKEVEGQILGTYVCIKGYHLVGKKTLFCNASKEWDNTTTECRLGHCPDPVLVNGEFSSSGPVNVSDKITFMCNDHYILKGSNRSQCLEDHTWAPPFPICKSRDCDPPGNPVHGYFEGNNFTLGSTISYYCEDRYYLVGVQEQQCVDGEWSSALPVCKLIQEAPKPECEKALLAFQESKNLCEAMENFMQQLKESGMTMEELKYSLELKKAELKAKLL

SEQ ID NO: 4 - protein, 59 aa, Homo sapiens
IQEAPKPECEKALLAFQESKNLCEAMENFMQQLKESGMTMEELKYSLELKKAELKAKLL

SEQ ID NO: 5 - protein, 116 aa, Homo sapiens
RDCDPPGNPVHGYFEGNNFTLGSTISYYCEDRYYLVGVQEQQCVDGEWSSALPVCKLIQEAPKPECEKALLAFQESKNLCEAMENFMQQLKESGMTMEELKYSLELKKAELKAKLL

SEQ ID NO: 6 - protein, 227 aa, synthetic construct
  REGION 1..227: note = IL-2 fused to the C-terminal region of C4BP (Hi2cb) includingthe signal peptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

SEQ ID NO: 7 - protein, 227 aa, synthetic construct
  REGION 1..227: note = mutated IL-2 (N88R) fused to the C-terminal region of C4BP(Hi2mcb), including the signal peptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

SEQ ID NO: 8 - protein, 5 aa, synthetic construct
  REGION 1..5: note = linker pattern
GGGGS

SEQ ID NO: 9 - protein, 207 aa, synthetic construct
  REGION 1..207: note = IL-2 fused to the C-terminal region of C4BP (Hi2cb) without thesignal peptide
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

SEQ ID NO: 10 - protein, 207 aa, synthetic construct
  REGION 1..207: note = mutated IL-2 (N88R) fused to the C-terminal region of C4BP(Hi2mcb) without the signal peptide
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLL

SEQ ID NO: 11 - protein, 113 aa, synthetic construct
  REGION 1..113: note = E06 VL Sequence
DIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIK

SEQ ID NO: 12 - protein, 123 aa, synthetic construct
  REGION 1..123: note = E06 VH Sequence
EVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSS

SEQ ID NO: 13 - protein, 251 aa, synthetic construct
  REGION 1..251: note = scFvE06
DIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIKGGGGSGGGGSGGGGSEVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSS

SEQ ID NO: 14 - protein, 15 aa, synthetic construct
  REGION 1..15: note = linker
GGGGSGGGGSGGGGS

SEQ ID NO: 15 - protein, 473 aa, synthetic construct
  REGION 1..473: note = IL-2-C4bp-scFvE06 (without peptide signal, and without HIS TAG)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLLGGGGSGGGGSGGGGSDIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIKGGGGSGGGGSGGGGSEVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSS

SEQ ID NO: 16 - protein, 493 aa, synthetic construct
  REGION 1..493: note = SEQ ID NO:16IL-2-C4bp-scFvE06 (with peptide signal, withoutHIS TAG)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLLGGGGSGGGGSGGGGSDIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIKGGGGSGGGGSGGGGSEVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSS

SEQ ID NO: 17 - protein, 473 aa, synthetic construct
  REGION 1..473: note = IL-2N88R-C4bp-scFvE06 (without peptide signal, and without HISTAG)
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLLGGGGSGGGGSGGGGSDIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIKGGGGSGGGGSGGGGSEVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSS

SEQ ID NO: 18 - protein, 493 aa, synthetic construct
  REGION 1..493: note = IL-2N88R-C4bp-scFvE06 (with peptide signal, without HIS TAG)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLLGGGGSGGGGSGGGGSDIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIKGGGGSGGGGSGGGGSEVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSS

SEQ ID NO: 19 - protein, 499 aa, synthetic construct
  REGION 1..499: note = IL-2-C4bp-scFvE06 (with peptide signal, with HIS TAG)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLLGGGGSGGGGSGGGGSDIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIKGGGGSGGGGSGGGGSEVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSSHHHHHH

SEQ ID NO: 20 - protein, 499 aa, synthetic construct
  REGION 1..499: note = IL-2N88R-C4bp-scFvE06 (with peptide signal, with HIS TAG)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLTGGGGSGGGGSGGGGSIQEAPKPECEKALLAFQESKNLCEAMENFMQQLKESGMTMEELKYSLELKKAELKAKLLGGGGSGGGGSGGGGSDIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIKGGGGSGGGGSGGGGSEVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSSHHHHHH